(19) United States
(12) Patent Application Publication
(10) Pub. No.: US 2022/0273660 A1
(43) Pub. Date: Sep. 1, 2022

(54) MEK INHIBITOR FOR TREATMENT OF STROKE

(71) Applicant: EDVINCE AB, Lund (SE)

(72) Inventor: Lars EDVINSSON, Lund (SE)

(21) Appl. No.: 17/630,287

(22) PCT Filed: Jul. 28, 2020
(86) PCT No.: PCT/EP2020/071209

§ 371 Date: Jan. 26, 2022

(30) Foreign Application Priority Data
Jul. 30, 2019 (EP) 19189069.8

(51) Int. Cl. A61K 31/519; A61K 31/4422; A61K 31/506; A61P 9/10

(52) U.S. Cl. CPC A61K 31/519; A61K 31/4422; A61K 31/506; A61P 9/10

(57) ABSTRACT

The present invention relates to a MEK inhibitor and compositions thereof, for use in the treatment of stroke, in particular treatment of subarachnoid haemorrhage (SAH)

### TECHNICAL FIELD

The present invention relates to a MEK inhibitor and its use as a medicament, for the treatment of stroke and associated conditions, including subarachnoid haemorrhage (SAH).

### BACKGROUND

Stroke is the second leading cause of death and a major cause of disability worldwide. Its incidence is increasing as a consequence of the aging population. Moreover, the incidence of stroke is increasing in young people in particular in low- and middle-income countries. Ischemic stroke is the more frequent type of stroke, while haemorrhagic stroke is responsible for more deaths and disability-adjusted life-years lost. Incidence and mortality of stroke differ between countries, geographical regions, and ethnic groups. In high-income countries mainly, improvements in prevention, acute treatment, and neurorehabilitation have led to a substantial decrease of the stroke burden over the past 30 years.

Aneurysmal subarachnoid haemorrhage (SAH), is a variant of haemorrhagic stroke causing around 5% of all stroke incidents, however with a striking 50% mortality rate. Survivors will often have cognitive impairments and reduced quality of life, making it a very debilitating disease. SAH is usually caused by the burst of an aneurysm, leading to a rapid leakage of arterial blood into the subarachnoid space, followed by a dramatic rise of the intracranial pressure (ICP) and a drop of the cerebral blood flow (CBF). This leads to oxygen and glucose deprivation of the brain resulting in cerebral ischemia and brain damage, often referred to as early brain injury. Delayed cerebral ischemia (DCI), is associated with secondary delayed brain injury and is comprised of various pathophysiological changes, including inflammation, oedema, and blood-brain barrier disruption. DCI is likely associated with remodelling and narrowing of the cerebral arteries, and especially the vascular hyperreactivity that are often referred to as delayed cerebral vasospasm (CVS), of which there is currently few treatment options.

Vascular contractility has therefore been the focus of many clinical and pre-clinical studies attempting to prevent the following DCI. This includes recent attempts to modulate acute vascular contractility, for example with endothelin receptor antagonists including the specific endothelin A (ET_{A}) and also endothelin B (ET_{B}) receptor antagonist clazosentan. These attempts have unfortunately not been successful.

Hence, there is a need in the art for the provision of new and superior stroke treatments to address the medical burden caused by stroke in its many forms.

### SUMMARY

The present inventor has surprisingly found that the MEK inhibitor trametinib and analogues thereof, display superior effects in an in vivo stroke model as compared to other potent MEK inhibitors.

In a first aspect, a MEK inhibitor of formula (I) is provided,

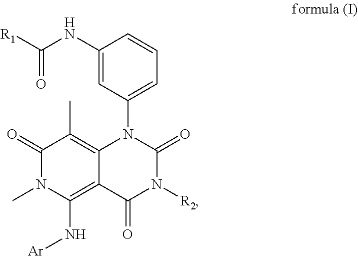

or a pharmaceutically acceptable salt thereof,

wherein;

R₁ is a C1-C6 alkyl, such as methyl,

R₂ is a C1-C6 alkyl, such as cyclopropyl,

Ar is selected from the group consisting of aryl, phenyl, and heteroaryl;

for use in the prevention or treatment of a stroke in a subject.

In a second aspect, the MEK inhibitor for the use of the present disclosure is of formula (II),

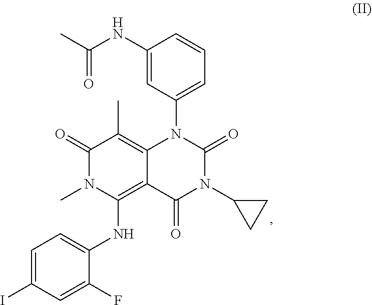 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof.

In a third aspect, use of a MEK inhibitor of formula (I) is provided, for:

a. reducing endothelin-1 induced contractility;

b. reducing the phenotypic change of a relaxant endothelin B receptor function to a contractile phenotype; and/or

c. improving neurological score, which may be evaluated by a subject's ability to traverse a rotating pole, after induced subarachnoid haemorrhage.

In a fourth aspect, a method of treating or reducing the risk of developing a stroke in a subject is provided, wherein the method comprises the steps of administering a MEK inhibitor of formula (I),

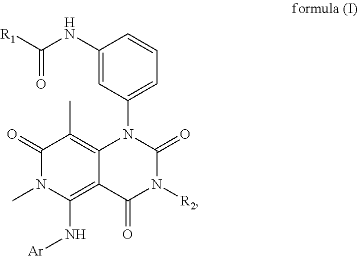

or a pharmaceutically acceptable salt thereof,

wherein;

R₁ is a C1-C6 alkyl, such as methyl,

R₂ is a C1-C6 alkyl, such as cyclopropyl,

Ar is selected from the group consisting of aryl, phenyl, and heteroaryl;

to a subject in need thereof, thereby treating or reducing the risk of developing a stroke.

In a fifth aspect, a composition is provided comprising, separately or together, the MEK inhibitor of formula (II),

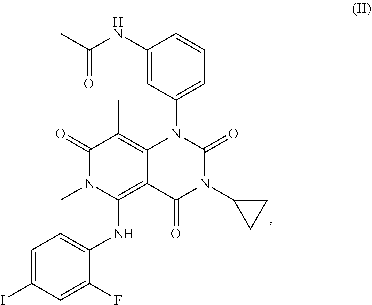 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof, and a further medicament.

### DESCRIPTION OF DRAWINGS

FIG. 1. Comparison of inhibitory capacity of nine different MEK1/2 inhibitors (1 μM), after 48 hours organ culture of the basilar artery. This results in a phenotypic alteration with upregulation of contractile ET_{B} receptors in the vascular smooth muscle cells. (A) Concentration-response curves to the ET_{B} specific agonist S6c, following incubation with 1 μM of different MEK1/2 inhibitors. In fresh vessels S6c results in relaxation of cerebral vessels or no effect but after organ culture the contractile phenotype appears and shows similar characteristics in different models of stroke. (B) Maximal contraction to 60 mM K⁺ following incubation with 1 μM of different MEK1/2 inhibitors. There is no significant difference between the vehicle (DMSO) and any of the antagonists. (C) Endothelium-induced dilation with significant increase in segments incubated TAK-733, trametinib and PD0325901 compared to vehicle (DMSO). Normally organ culture shows reduced endothelial function but these inhibitors protected this detrimental effect. Data are shown as mean±SEM with statistics by two-way ANOVA followed by a Holm-Sidak's multiple comparison test. * P<0.05, ** P<0.01 compared to DMSO. n=4-15.

FIG. 2. Concentration-response curves of selected highly potent MEK1/2 inhibitors. Concentration-response curves of selected inhibitors based on the (A) Emax of S6c, (B) maximal contraction to 60 mM K⁺ and (C) endothelium function evaluated buy the addition of 10⁻⁵ M carbachol on arteries precontracted with 3·10-7 M 5-HT. Data are shown as mean±SEM, n=4.

FIG. 3. Effect of trametinib and PD0325901 on pathways regulating vasomotion after 48 hours organ culture of basilar artery. Arteries from OC in the presence of DMSO control (n=5), 1 μM of trametinib (n=6) or PD0325901 (n=5) were precontracted with U46619 (1-3·10⁻⁷ M) or K⁺ (41 mM) and cumulative concentration-response curves was performed by adding (A) Carbachol (10⁻¹⁰-10⁻⁵ M), (B) SNP (10⁻¹¹-10⁻⁴ M) or (C) CGRP (10⁻¹²-10⁻⁷ M). (D) Cumulative concentration-response curves with Ca²⁺ (0.0125-3 mM) after an ET-1 precontraction (10⁻⁷ M) following organ culture with DMSO control (n=15), 1 μM of trametinib (n=4) or PD0325901 (n=4). Data is shown as mean±SEM with statistics by two-way ANOVA, followed by Holm-Sidak's multiple comparison test */#P<0.05, **/##P<0.01, ***/###P<0.001. For normalised data the Geisser-Greenhouse correction for sphericity was applied.

FIG. 4. Effect of trametinib and PD0325901 treatment on contractile responses to 60 mM K⁺ and on the endothelium function after SAH and sham surgery in rat. (A) Emax, K⁺ (Nm⁻¹) induced by 60 mM K⁺. (B) Endothelium function. Data includes basilar arteries below 2.0 mN cut-off for comparison of all arteries; sham+vehicle (n=10), SAH (n=11), SAH+vehicle (n=27), trametinib (n=13) and PD0325901 (n=12). Horizontal dotted black line shows Total mean of all n (n=73). Data is shown as mean±SEM, in this figure ‘n’ equals individual arterial segments. Statistics was done by one-way ANOVA followed by Holm-Sidak's multiple comparison test. * P<0.05, ** P<0.01.

FIG. 5. Effect of trametinib and PD0325901 treatment on contractile responses to endothelin-1 after SAH or sham surgery in rat. Cumulative concentration-response curves to ET-1 (10⁻¹⁴-10⁻⁷ M) of basilar arteries. (A) ET-1 (Nm⁻¹) and (B) ET-1 (% of ET-1max), for sham+vehicle (n=5), SAH (n=5), SAH+vehicle (n=12), SAH+trametinib (n=6) and SAH+PD0325901 (n=6). Data are shown as mean±SEM, with statistics by two-way ANOVA followed by Holm-Sidak's multiple comparison test. * P=<0.05. ET-1max normalised data used Geisser-Greenhouse correction for sphericity and ET-1 curves are a biphasic non-linear regression curve fit.

FIG. 6. Effect of trametinib and PD0325901 treatment on VDCC independent ET-1-induced contractions after SAH or sham surgery in rat. Basilar arteries were precontracted with ET-1 (10⁻⁷ M) followed by cumulative concentration-response curves with Ca²⁺ (Nm⁻¹); sham+vehicle (n=5), SAH (n=5), SAH+vehicle (n=13), SAH+trametinib (n=7) and SAH+PD0325901 (n=6). Data is shown as mean±SEM, with statistics by two-way ANOVA followed by Holm-Sidak's multiple comparison test. */#/$ P=<0.05.

FIG. 7. Effect of trametinib and PD0325901 treatment on neurologic function. Rotating pole in vivo test at 10 rpm for; (A) Pre-SAH, (B) 24 hours post-SAH and (C) 48 hours post-SAH. Scored with 4 counts per animal, i.e. 2 scores for left- and right rotation, respectively; Low=Unable to traverse in one try; High=Able to traverse in one try. Sham+vehicle (n=5), SAH (n=9), SAH+vehicle (n=14), SAH+trametinib (n=7) and SAH+PD0325901 (n=6). All animals, excluding sham+vehicle group, were exposed to experimental SAH. Statistics were done by a Fischer's exact test, two-sided, 95% CI. * P<0.05; ** P<0.01; *** P<0.001.

FIG. 8. Effect of i.p. trametinib treatment after SAH surgery in rat. (A) Emax, for K⁺ (Nm⁻¹) induced by 60 mM e for SAH+i.p. vehicle (n=10), i.p. trametinib (n=12). Data is shown as mean±SEM. In this figure ‘n’ equals individual arterial segments. (B) Cumulative concentration-response curves to ET-1 (10⁻¹⁴-10⁻⁷ M) of basilar arteries normalized to 60 mM e from animals treated with SAH+i.p. vehicle (n=5) or SAH+i.p. trametinib (n=6). Data are shown as mean±SEM, with statistics by two-way ANOVA followed by Holm-Sidak's multiple comparison test. *** P=<0.001. ET-1 curves are a biphasic non-linear regression curve fit. The right panel show the rotating pole tests at 10 rpm for neurological function at (C) 24 hours post-SAH and (D) 48 hours post-SAH. Scored with 4 counts per animal, i.e. 2 scores for left- and right rotation, respectively; Low=Unable to traverse in one try; High=Able to traverse in one try. Statistics were done by a Fischer's exact test, two-sided, 95% Cl. * P<0.05.

FIG. 9. Table with regimens for intrathecal and intraperitoneal treatment.

FIG. 10. Table with curve fits and comparison of EC₅₀ values.

FIG. 11. Table with surgical data for intrathecal and intraperitoneal treatments.

FIG. 12. Table with data from a rotating pole test.

FIG. 13. Overview of the inhibitors used in the present study.

FIG. 14. Physiological parameters: Body weight, mean arterial blood pressure (MABP), pH, CO₂ pressure (pCO₂) and O₂ pressure (pO₂) of animals subjected to experimentally induced SAH (intracisternal injection of 300 μL autologous blood) or sham-operation (control) in female rats. Values are means±SEM, n=16-18 rats in each group.

FIG. 15. Contractile effects of 5-CT and ET-1 in cerebral arteries. Pharmacological parameters for contractile responses of basilar artery (BA) and middle cerebral artery (MCA) to 5-CT (5HT_{1B/D} agonist) and ET-1 (ET_{A/B} agonist) 2 days after experimentally induced SAH (injection of either 250 or 300 μL autologous blood) or sham operation (control) in female rat. Contractile responses were characterized by maximum contractile response (Eₘₐₓ) values, expressed as percentage of 60 mM K⁺ induced contraction (K⁺ response), and values of the negative logarithm of the molar concentration that produces half maximum contraction (pEC₅₀). For biphasic concentration-contraction curves, Eₘₐₓ and pEC₅₀ values for each of the two phases are provided. Values are means±SEM, n=numbers of rats.

FIG. 16. Intracranial pressure and relative cerebral blood flow pre- during- and post-surgery. Intracranial pressure (ICP) and relative cerebral blood flow (rCBF) changes of animals during the surgery of experimentally induced SAH (300 μL of autologous blood injection) or sham-operation (control) in female rats. Values are means±SEM, n=16-18 rats in each group.

FIG. 17. Effect of ovariectomy on vasocontractile responses of middle cerebral arteries after transient middle cerebral artery occlusion. (A) Contraction induced by sarafotoxin (S6c), a selective endothelin B receptor agonist. a: P<0.01 compared to intact non-occluded. b: P<0.01 compared to ovariectomized (OVX) non-occluded. (B) Contraction induced by 5-carboxamidotryptamine (5-CT), a non-selective 5-hydroxytryptamine receptor agonist. A: P<0.01 intact non-occluded compared to occluded. B: P<0.01 OVX non-occluded compared to occluded. (C) Contraction induced by angiotensin II (Ang II) in the presence of an angiotensin II receptor type 2 blocker resulting in an angiotensin II receptor type 1-mediated response. Contraction is expressed as percentage of maximum potassium-mediated contraction (mean±SEM). The experiments were performed in the presence of N-nitro-L-arginine methyl ester (100 μM) and indomethacin (10 μM) to block nitric oxide synthase and the production of prostaglandins, respectively. *P<0.05. MCA: middle cerebral artery

FIG. 18. Effect of hormone replacement in ovariectomized females on vasocontractile responses of middle cerebral arteries after transient middle cerebral artery occlusion. (A) Contraction induced by sarafotoxin 6c (S6c) a selective endothelin B receptor agonist. As there were no significant differences among the responses in non-occluded arteries from the different groups, the data were combined and the mean values are shown here. (B) Contraction induced by 5-carboxamidotryptamine (5-CT), a non-selective 5-hydroxytryptamine receptor agonist. (C) Contraction induced by angiotensin II (Ang II) in the presence of an angiotensin II receptor type 2 receptor blocker resulting in angiotensin II receptor type 1 receptor-mediated response. Contraction is expressed as percentage of maximum potassium-mediated contraction (mean±SEM). The experiments were performed in the presence of N-nitro-L-arginine methyl ester (100 μM) and indomethacin (10 μM) to block nitric oxide synthase and the production of prostaglandins, respectively. *P<0.05, *** <0.001. OVX: Ovariectomized, E: 17β-estradiol, P: progesterone

FIG. 19. Endothelin B receptor mediated contraction of cultured middle cerebral arteries from ovariectomized females. Contraction induced by sarafotoxin 6c (S6c), a selective endothelin ETB receptor agonist, in middle cerebral arteries subjected to 24 h organ culture. Comparison between middle cerebral arteries from intact females, ovariectomized (OVX) or OVX treated with 17β-estradiol (OVX+E). Contraction is expressed as percentage of maximum potassium-mediated contraction (mean±SEM).

FIG. 20. Table 1. Comparison of Emax values for contractile responses in MCAs from intact females, ovariectomized females and males after tMCAO. Maximum contractile response (Emax) induced by sarafotoxin (S6c), 5-carboxamidotryptamin (5-CT) and Angiotensin II (Ang II) in occluded and non-occluded middle cerebral arteries isolated 48 hours after transient middle cerebral artery occlusion (tMCAO). Contraction is expressed as percentage of maximum potassium-mediated contraction (mean±SD). Intact: females with intact ovaries, OVX: ovariectomized females. *P<0.05 compared to non-occluded. **P<0.01 compared to non-occluded. ns—no significant differences between occluded and non-occluded. a,b—lower response than intact occluded (P<0.05). ns=no statistically significant difference compared to non-occluded.

FIG. 21. In vitro experiments, freshly isolated MCAs (controls) showed no contractile response to the ET_{B} receptor agonist S6c. (A) After 48 h of OC, S6c yielded a strong contractile response in MCAs incubated with vehicle. However, co-incubation with trametinib (GSK1120212) significantly inhibited the S6c-induced contraction 48 h after OC in a concentration-dependent manner. (B) The maximal contraction (Eₘₐₓ) induced by S6c, in all groups. (C) 0.1 μM of trametinib (GSK1120212) confirmed the inhibitory effect on increased ET-1-induced vasoconstriction

FIG. 22. In vivo experiments, the effect of the trametinib (depicted as GSK) treatment on SAH-induced increased ET-1 mediated vasoconstriction, two different treatment approaches were used. (A) intraperitoneal administration of 1 mM trametinib at 1 and 24 h or (B) intraperitoneal administration of 1 mM trametinib at 6 and 24 h post-SAH. (C) Flow cytometry: the enhanced contractile responses observed after the 6 h post-SAH treatment was verified with protein analyses using flow cytometry. There was a significant increase of SMC expressing ET_{B} receptor after SAH (vehicle) (74.2%±12.2%; n=7) compared to sham (61.4%±10.2%; n=6).

### DETAILED DESCRIPTION

Terms and Definitions

The terms “treatment” and “treating” as used herein refer to the management and care of a patient for the purpose of combating a condition, disease or disorder. The term is intended to include the full spectrum of treatments for a given condition from which the patient is suffering. The patient to be treated is preferably a mammal, in particular a human being. Treatment of animals, such as mice, rats, dogs, cats, horses, cows, sheep and pigs, is, however, also within the scope of the present context. The patients to be treated can be of various ages.

The term “global ischemia” as used herein refers to ischemia affecting a wider area of the brain and usually occurs when the blood supply to the brain has been drastically reduced or stops. This is typically caused by a cardiac arrest.

The term “focal ischemia” as used herein refers to ischemia confined to a specific area of the brain. It usually occurs when a blood clot has blocked an artery in the brain. Focal ischemia can be the result of a thrombus or embolus.

The term “traumatic brain injury” (TBI), also known as an intracranial injury, is an injury to the brain caused by an external force. TBI can be classified based on severity, mechanism (closed or penetrating head injury) or other features (e.g., occurring in a specific location or over a widespread area). TBI can result in physical, cognitive, social, emotional and behavioral symptoms, and outcomes can range from complete recovery to permanent disability or death

MEK Inhibitors

In one embodiment, a MEK inhibitor of formula (I) is provided,

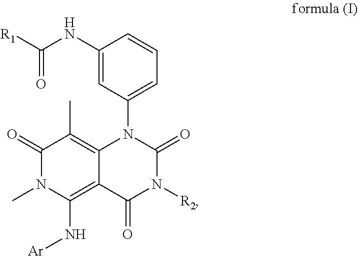

or a pharmaceutically acceptable salt thereof,

wherein;

R₁ is a C1-C6 alkyl, such as methyl,

R₂ is a C1-C6 alkyl, such as cyclopropyl,

Ar is selected from the group consisting of aryl and heteroaryl;

for use in the prevention or treatment of a stroke in a subject.

In one embodiment of the present disclosure, a MEK inhibitor of formula (I) is provided, wherein R₁ is a C1-C3 alkyl. In a preferred embodiment, R₁ is a linear C1-C3 alkyl. In a further preferred embodiment, R₁ is methyl or ethyl. In the most preferred embodiment, R₁ is methyl.

In one embodiment of the present disclosure, a MEK inhibitor of formula (I) is provided, wherein R₂ is C2-C4 alkyl. In a further embodiment, R₂ is C3 or C4 cycloalkyl. In a preferred embodiment, R₂ is cyclopropyl.

In one embodiment of the present disclosure, a MEK inhibitor of formula (I) is provided, wherein Ar is phenyl or substituted phenyl. In a further embodiment, Ar is substituted phenyl. In a preferred embodiment, Ar is 2-fluoro-4-iodophenyl.

In one embodiment of the present disclosure, a MEK inhibitor of formula (I) is provided, wherein R₁ is a C1-C3 alkyl, R₂ is C2-C4 alkyl, and Ar is substituted phenyl.

In a preferred embodiment of the present disclosure, a MEK inhibitor of formula (I) is provided, wherein R₁ is methyl or ethyl, R₂ is C3 or C4 cycloalkyl, and Ar is substituted phenyl.

In one embodiment, the MEK inhibitor is provided for the use as defined herein, wherein the MEK inhibitor is of formula (II),

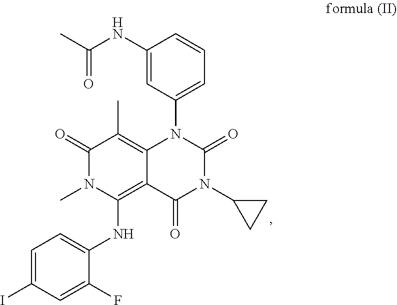 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof.

In one embodiment, use of a MEK inhibitor of formula (I) is provided, for:

a. reducing endothelin-1 induced contractility;

b. reducing the increased contractile endothelin B receptor function; and/or

c. improving neurological score, which may be evaluated by a subject's ability to traverse a rotating pole, after induced subarachnoid haemorrhage.

In one embodiment, a method of treating or reducing the risk of developing a stroke in a subject is provided, wherein the method comprises the steps of administering a MEK inhibitor of formula (I),

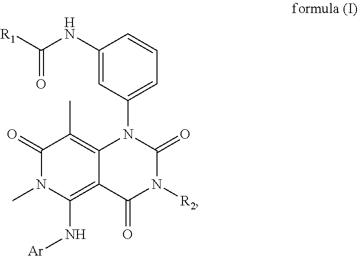

or a pharmaceutically acceptable salt thereof,

wherein;

R₁ is a C1-C6 alkyl, such as methyl,

R₂ is a C1-C6 alkyl, such as cyclopropyl,

Ar is selected from the group consisting of aryl, phenyl, and heteroaryl;

to a subject in need thereof, thereby treating or reducing the risk of developing a stroke.

Substituents

“Alkyl” refers to a straight, branched, or cyclic hydrocarbon chain radical consisting of carbon and hydrogen atoms, containing no unsaturation, and may be straight or branched, substituted or unsubstituted. In some preferred embodiments, the alkyl group may consist of 1 to 12 carbon atoms, e.g. 1 carbon atom, 2 carbon atoms, 3 carbon atoms, 4 carbon atoms etc., up to and including 12 carbon atoms. Exemplary alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of any suitable substituents. An alkyl group can be mono-, di-, tri- or tetra-valent, as appropriate to satisfy valence requirements.

The term “alkylene,” by itself or as part of another substituent, means a divalent radical derived from an alkyl moiety, as exemplified, but not limited, by —CH₂CH₂CH₂CH₂—.

By “cycloalkyl” is meant an alkyl group specifically comprising a cyclic moiety. Exemplary cycloalkyl groups include, but are in no way limited to, cyclopropyl, cyclobutyl, cyclopentyl, or cyclohexyl.

By “substituted” means replacement of a hydrogen atom from a parent moiety and replacement by another chemical group. Substituents considered are, but not limited to: alkyl groups such as C1-C6 alkyl; alkoxy groups such as C1-C6 alkoxy; halogen atoms such as —F, —Cl, —Br, or —I; —CN; —NO; —NO₂; —SO₂H; —SO₃H; —CO₂H; hydroxy; amino; thiol; aryl; heteroaryl; or acyl.

By “aryl” is meant both unsubstituted and substituted aryl groups.

By “heteroaryl” is meant both unsubstituted and substituted heteroaryl groups.

Stroke in General

Strokes can be classified into at least two major categories: ischemic and hemorrhagic. Ischemic strokes are caused by interruption of the blood supply to the brain, while hemorrhagic strokes result from the rupture of a blood vessel or an abnormal vascular structure. About 87% of strokes are ischemic, the rest being hemorrhagic. According to the present disclosure, a stroke may also include a transient ischemic attack (TIA) or can be the result of a heart stop or dramatic lowering of systemic blood pressure by other means, e.g. heart fibrillation.

In one embodiment of the present disclosure, the stroke is selected from the group consisting of: ischemic stroke, haemorrhagic stroke, and transient ischemic attack.

In one embodiment of the present disclosure, the stroke is selected from the group consisting of: global ischemia and focal ischemia.

In one embodiment, the MEK inhibitor is administered to the subject before it has been determined if the subject suffers from an acute ischemic stroke or a haemorrhagic stroke.

Ischemic Stroke

In an ischemic stroke, blood supply to part of the brain is decreased, leading to dysfunction of the brain tissue in that area. There are four main reasons why this might happen:

1. Thrombosis (obstruction of a blood vessel by a blood clot forming locally)

2. Embolism (obstruction due to an embolus from elsewhere in the body),

3. Systemic hypoperfusion (general decrease in blood supply, e.g., in shock)

4. Cerebral venous sinus thrombosis.

But the stroke may also result from a sudden drop in blood pressure or heart stop, rupture of a cerebral artery or arteriole, or a combination thereof.

In one embodiment of the present disclosure, the ischemic stroke results from Traumatic Brain Injury (TBI) also known as an intracranial injury.

In one embodiment of the present disclosure, the ischemic stroke results from an embolism, thrombosis, systemic hypoperfusion, cerebral venous sinus thrombosis, a sudden drop in blood pressure or heart stop, rupture of a cerebral artery or arteriole, or a combination thereof.

Haemorrhagic Stroke

There are at least two main types of hemorrhagic stroke:
“Intracerebral hemorrhage, which is basically bleeding within the brain itself (when an artery in the brain bursts, flooding the surrounding tissue with blood), due to either intraparenchymal hemorrhage (bleeding within the brain tissue) or intraventricular hemorrhage (bleeding within the brain's ventricular system).
Subarachnoid hemorrhage (SAH), which is basically bleeding that occurs outside of the brain tissue but still within the skull, and precisely between the arachnoid mater and pia mater (the delicate innermost layer of the three layers of the meninges that surround the brain), usually due to the rupture of a cerebral artery or an arterial mailformation.

The above two main types of hemorrhagic stroke are also two different forms of intracranial hemorrhage, which is the accumulation of blood anywhere within the cranial vault.

Hemorrhagic strokes may occur on the background of alterations to the blood vessels in the brain, such as cerebral amyloid angiopathy, cerebral arteriovenous malformation and an intracranial aneurysm, which can cause intraparenchymal or subarachnoid hemorrhage.

In addition to neurological impairment, hemorrhagic strokes usually cause specific symptoms (for instance, subarachnoid hemorrhage classically causes a severe headache known as a thunderclap headache) or reveal evidence of a previous head injury.

In one embodiment, the MEK inhibitor as defined herein is provided for use in prevention or treatment of a stroke, which is a haemorrhagic stroke that results from intracerebral haemorrhage, subarachnoid haemorrhage, or a combination thereof.

In one embodiment, the intracerebral haemorrhage is intraparenchymal, intraventricular, or a combination thereof.

In one embodiment, the stroke results from subarachnoid haemorrhage.

Delayed Cerebral Ischemia (DCI)

Delayed cerebral ischemia may occur days after subarachnoid hemorrhage and represents a potentially treatable cause of morbidity for approximately one-third of those who survive the initial hemorrhage. While vasospasm has been traditionally linked to the development of cerebral ischemia several days after subarachnoid hemorrhage, emerging evidence reveals that delayed cerebral ischemia is part of a much more complicated post-subarachnoid hemorrhage syndrome. The development of delayed cerebral ischemia involves early arteriolar vasospasm with microthrombosis, perfusion mismatch and neurovascular uncoupling, spreading depolarizations, and inflammatory responses that begin at the time of the hemorrhage and evolve over time, culminating in cortical infarction.

In one embodiment, the MEK inhibitor as defined herein is used in treatment or prevention of delayed cerebral ischemia (DCI).

In one embodiment, the DCI presents with inflammation, oedema, delayed cerebral vasospasm (CVS), blood-brain barrier disruption and/or increase in contractile receptor expression, such as those for endothelin, angiotensin, serotonin and thromboxane or prostaglandins.

Surgery and Combination Therapy

In one embodiment, the MEK inhibitor is administered to the subject without surgery prior to, concurrent with, or subsequent to the administration.

In one embodiment, the MEK inhibitor is administered to the subject prior to, concurrent with, or subsequent to thrombectomy.

In one embodiment, the MEK inhibitor is administered to the subject prior to, concurrent with, or subsequent to thrombolysis.

In one embodiment, the MEK inhibitor of the present disclosure is administered to the subject prior to, concurrent with, or subsequent to a surgical procedure selected from the group consisting of: coiling and clipping.

The procedure “coiling” or “endovascular coiling” is a procedure performed to block blood flow from an aneurysm (a weakened area in the wall of an artery). Endovascular coiling is a minimally invasive technique, which means an incision in the skull is not required to treat the brain aneurysm. Rather, a catheter is used to reach the aneurysm in the brain. During endovascular coiling, a catheter is passed through the groin up into the artery containing the aneurysm. Platinum coils are then released. The coils induce clotting (embolization) of the aneurysm and, in this way, prevent blood from getting into it.

The procedure “clipping” or “microsurgical clipping” is a technique that blocks the blood supply to an aneurysm using a metal clip. The procedure is well-known to a person of skill in the art.

In one embodiment, the MEK inhibitor is administered to the subject prior to, concurrent with, or subsequent to a neuroradiological procedure.

Most “neuroradiological procedures” or “interventional neuroradiology procedures” begin with insertion of a catheter into the femoral artery, which is a large artery located in the groin. The catheter, which is a long, flexible hollow tube, is threaded over a guide wire up into the aorta, the main artery supplying the body, and then into the neck vessel leading to the blocked brain artery. Images of the artery (also known as angiography) are then taken using a radiographic contrast dye similar to the one used for CT angiography. These pictures allow the interventional neuroradiologist to identify the site of occlusion and plan the intervention. A smaller catheter (micro catheter) is then placed through the initial catheter and past the occlusive clot.

There are two main approaches to clot removal: whole-clot retrieval (or thrombectomy) and clot aspiration. In the first technique, the clot retrieval device is placed through the micro catheter, and opened across the clot. The device which traps the clot is then removed. The second technique, clot aspiration, involves fragmentation and suction of the clot. This is performed using catheters larger than traditional micro catheters, which provide increased suction power. Thrombectomy and aspiration techniques are often used in combination. In addition to these mechanical approaches, many interventional neuroradiologists also use local TPA infusion into the clot to help dissolve it.

In one embodiment of the present disclosure, the MEK inhibitor reduces or prevents reperfusion damage resulting from the neuroradiological procedure.

Reperfusion injury, sometimes called ischemia-reperfusion injury (IRI) or reoxygenation injury, is the tissue damage caused when blood supply returns to tissue (re-+perfusion) after a period of ischemia or lack of oxygen (anoxia or hypoxia). The absence of oxygen and nutrients from blood during the ischemic period creates a condition in which the restoration of circulation results in inflammation and oxidative damage through the induction of oxidative stress rather than (or along with) restoration of normal function.

In one embodiment, a composition is provided comprising, separately or together, the MEK inhibitor of formula (II),

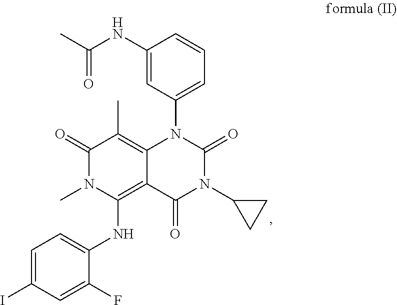 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof, and a further medicament.

In one embodiment, the further medicament is selected from the group consisting of: a calcium channel blocker, such as Nimodipine, and an endothelin receptor (ET) receptor blocker, such as clazosentan.

In one embodiment the further medicament is selected from the group Calcium channel blockers.

In one embodiment the further medicament is selected from the sub-class Dihydropyridine of Calcium channel blockers.

In one embodiment the further medicament is selected from Dihydropyridine such as Amlodipine (Norvasc), Aranidipine (Sapresta), Azelnidipine (Calblock), Barnidipine (HypoCa), Benidipine (Coniel), Cilnidipine (Atelec, Cinalong, Siscard),Clevidipine (Cleviprex), Efonidipine (Landel), Felodipine (Plendil), Isradipine (DynaCirc, Prescal), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas).

In one embodiment, a kit of parts is provided comprising;

a MEK inhibitor as defined herein; and

a further medicament as defined herein;

wherein the MEK inhibitor and the further medicament are formulated for simultaneous or sequential use; and optionally instructions for use.

Compositions and Administration

In one embodiment the present invention relates a pharmaceutical composition comprising an effective amount of a MEK inhibitor a further medicament.

While the MEK inhibitor as disclosed herein may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In one embodiment, the disclosure provides compositions comprising the MEK inhibitor as defined herein, or a pharmaceutically acceptable salt or derivative thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof. In a further embodiment, the invention provides pharmaceutical compositions or compositions comprising more than one compound or prodrug for use according to the disclosure, such as two different compounds or prodrugs for use according to the disclosure.

Compositions of the disclosure may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the disclosure, which matrices may be in form of shaped articles, e.g. films or microcapsules. Another suitable example is nanoparticles.

In one embodiment, the MEK inhibitor for use as defined herein is administered orally, intrathecally, intraperitoneally, intraocularly, or intravenously.

In one embodiment, the MEK inhibitor for use as defined herein is administered intranasally.

In one embodiment, the MEK inhibitor is administered intravenously. In one embodiment, the MEK inhibitor is administered to the subject up to 6 hours subsequent to the onset of the stroke, such as up to 1 hour, such as up to 2 hours, such as up to 3 hours, such as up to 4 hours, such as up to 5 hours subsequent to the onset of the stroke. In one embodiment, the treatment is continued past the first dose of MEK inhibitor for up to 3 days subsequent to the onset of the stroke.

In one embodiment, the MEK inhibitor is administered one or more times daily for up to 3 days subsequent to the onset of the stroke.

In one embodiment, the MEK inhibitor is administered to the subject in combination with a neuroprotective agent. Treatment of the subject by the MEK inhibitor may be discontinued 1, 2 or 3 days subsequent to the onset of the stroke, while treatment with the neuroprotective agent is continued. In one embodiment, the neuroprotective treatment is continued for one or more months.

Subjects

The subject according to the present disclosure may be any subject suffering or about to suffer from a stroke. Preferably, the subject is a human subject, such as a patient. In one embodiment, the subject is a human subject without any history of past strokes. In one embodiment, the human subject has previously suffered from stroke.

Items
1. A MEK inhibitor of formula (I),

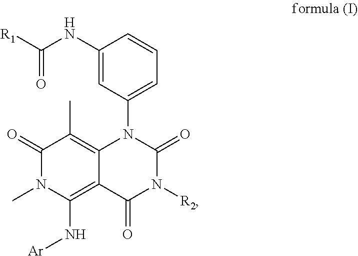

or a pharmaceutically acceptable salt thereof,
wherein;
R₁ is a C1-C6 alkyl, such as methyl,
R₂ is a C1-C6 alkyl, such as cyclopropyl,
Ar is selected from the group consisting of aryl and heteroaryl;
for use in the prevention or treatment of a stroke in a subject.
2. The MEK inhibitor according to any one of the preceding items, wherein R₁ is a C1-C3 alkyl.
3. The MEK inhibitor according to any one of the preceding items, wherein R₁ is a linear C1-C3 alkyl.
4. The MEK inhibitor according to any one of the preceding items, wherein R₁ is methyl or ethyl.
5. The MEK inhibitor according to any one of the preceding items, wherein R₁ is methyl.
6. The MEK inhibitor according to any one of the preceding items, wherein R₂ is C2-C4 alkyl.
7. The MEK inhibitor according to any one of the preceding items, wherein R₂ is C3 or C4 cycloalkyl.
8. The MEK inhibitor according to any one of the preceding items, wherein R₂ is cyclopropyl.
9. The MEK inhibitor according to any one of the preceding items, wherein Ar is phenyl or substituted phenyl.
10. The MEK inhibitor according to any one of the preceding items, wherein Ar is substituted phenyl.
11. The MEK inhibitor according to any one of the preceding items, wherein Ar is 2-fluoro-4-iodophenyl.
12. The MEK inhibitor according to any one of the preceding items, wherein R₁ is a C1-C3 alkyl, R₂ is C2-C4 alkyl, and Ar is substituted phenyl.
13. The MEK inhibitor according to any one of the preceding items, wherein R₁ is methyl or ethyl, R₂ is C3 or C4 cycloalkyl, and Ar is substituted phenyl.
14. The MEK inhibitor for use according to any one of the preceding items, wherein the MEK inhibitor is of formula (II),

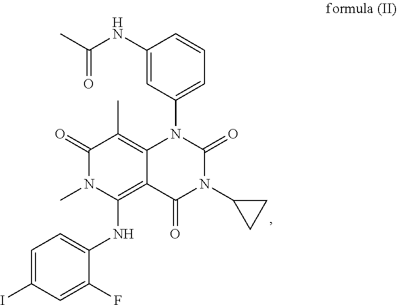 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof.
15. The MEK inhibitor for use according to any one of the preceding items, wherein the stroke is selected from the group consisting of: ischemic stroke, haemorrhagic stroke, and transient ischemic attack.
16. The MEK inhibitor for use according to any one of the preceding items, wherein the stroke is selected from the group consisting of: global ischemia and focal ischemia.
17. The MEK inhibitor for use according to any one of the preceding items, wherein the ischemic stroke results from an embolism, thrombosis, systemic hypoperfusion, cerebral venous sinus thrombosis, a sudden drop in blood pressure or heart stop, rupture of a cerebral artery or arteriole, or a combination thereof.
18. The MEK inhibitor for use according to any one of the preceding items, wherein the haemorrhagic stroke results from intracerebral haemorrhage, subarachnoid haemorrhage, or a combination thereof.
19. The MEK inhibitor for use according to any one of the preceding items, wherein the intracerebral haemorrhage is intraparenchymal, intraventricular, or a combination thereof.
20. The MEK inhibitor for use according to any one of the preceding items, wherein the stroke results from subarachnoid haemorrhage.
21. The MEK inhibitor for use according to any one of the preceding items, wherein the stroke is a delayed cerebral ischemia (DCI).
22. The MEK inhibitor for use according to any one of the preceding items, wherein the stroke results from traumatic brain injury (TBI).
23. The MEK inhibitor for use according to any one of the preceding items, wherein the DCI presents with inflammation, oedema, delayed cerebral vasospasm (CVS), blood-brain barrier disruption and/or increase in contractile receptor expression, such as those for endothelin, angiotensin, serotonin and thromboxane or prostaglandins.
24. The MEK inhibitor for use according to any one of the preceding items, wherein the MEK inhibitor is administered to the subject without surgery prior to, concurrent with, or subsequent to the administration.
25. The MEK inhibitor for use according to any one of the preceding items, wherein the MEK inhibitor is administered to the subject prior to, concurrent with, or subsequent to thrombectomy.
26. The MEK inhibitor for use according to any one of the preceding items, wherein the MEK inhibitor is administered to the subject prior to, concurrent with, or subsequent to thrombolysis.
27. The MEK inhibitor for use according to any one of the preceding items, wherein the MEK inhibitor is administered to the subject prior to, concurrent with, or subsequent to a surgical procedure selected from the group consisting of: coiling and clipping.
28. The MEK inhibitor for use according to any one of the preceding items, wherein the MEK inhibitor is administered to the subject prior to, concurrent with, or subsequent to a neuroradiological procedure.
29. The MEK inhibitor for use according to any one of the preceding items, wherein the MEK inhibitor reduces or prevents reperfusion damage resulting from the neuroradiological procedure.
30. The MEK inhibitor for use according to any one of the preceding items, wherein the MEK inhibitor is administered to the subject before it has been determined if the subject suffers from an acute ischemic stroke or a haemorrhagic stroke.
31. The MEK inhibitor for use according to any one of the preceding items, wherein the MEK inhibitor is administered orally, intrathecally, intraperitoneally, intraocularly, or intravenously.
32. The MEK inhibitor for use according to any one of the preceding items, wherein the MEK inhibitor is administered intravenously.
33. The MEK inhibitor for use according to any one of the preceding items, wherein the MEK inhibitor is administered to the subject up to 6 hours subsequent to the onset of the stroke, such as up to 1 hour, such as up to 2 hours, such as up to 3 hours, such as up to 4 hours, such as up to 5 hours subsequent to the onset of the stroke.
34. The MEK inhibitor for use according to any one of the preceding items, wherein the MEK inhibitor is administered one or more times daily for up to 3 days subsequent to the onset of the stroke.
35. The MEK inhibitor for use according to any one of the preceding items, wherein the subject is a human subject.
36. Use of a MEK inhibitor of formula (I) as defined in any one of the preceding items, for:
   a. reducing endothelin-1 induced contractility;
   b. increasing endothelin B receptor function; and/or
   c. improving neurological score, which may be evaluated by a subject's ability to traverse a rotating pole, after induced subarachnoid haemorrhage.
37. A method of treating or reducing the risk of developing a stroke in a subject, wherein the method comprises the steps of administering a MEK inhibitor of formula (I),

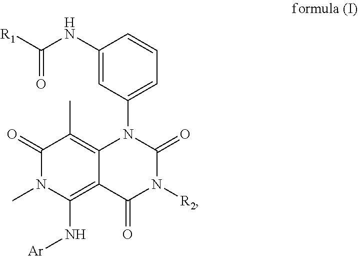

or a pharmaceutically acceptable salt thereof,
wherein;
R₁ is a C1-C6 alkyl, such as methyl,
R₂ is a C1-C6 alkyl, such as cyclopropyl,
Ar is selected from the group consisting of aryl, phenyl, and heteroaryl;
to a subject in need thereof, thereby treating or reducing the risk of developing a stroke.
38. A composition comprising, separately or together, the MEK inhibitor of formula (II),

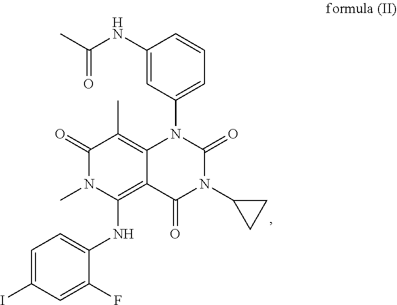 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof, and a further medicament.
39. The composition according to any one of the preceding items, wherein the further medicament is selected from the group consisting of: a calcium channel blocker, such as Nimodipine, and an endothelin receptor (ET) receptor blocker, such as clazosentan.
40. A kit of parts comprising;
   a MEK inhibitor as defined in any one of the preceding items; and
   a further medicament as defined in any one of the preceding items;
   wherein the MEK inhibitor and the further medicament are formulated for simultaneous or sequential use; and optionally instructions for use.

### EXAMPLES

### Example 1

### Organ Culture and Concentration-Response Curves to a Selection of MEK1/2 Inhibitors

Materials and Methods

Husbandry, Housing and Ethics

110 Sprague-Dawley rats (NTac:SD), obtained from Taconic (Denmark), were maintained at a 12/12-h light-dark cycle (with light beginning at 7 a.m.) and housed at a constant temperature (22±2° C.) and humidity (55±10%), with food and water ad libitum. Rats were generally housed in Eurostandard cages (Type VI with 123-Lid) 2-6 together and single housed (Type III with 123-Lid) after the surgical procedure. 52 male Sprague-Dawley rats (298-370 g) was used for surgical procedures and were approved by the Danish Animal Experimentation Inspectorate (license no. 2016-15-0201-00940). The animal work was performed at the Glostrup Research park, Rigshospitalet-Glostrup, Denmark.

Harvest and Organ Culture of Cerebral Arteries (Ex Vivo Model)

Rats were sedated with O₂/CO₂ (30/70%) and sacrificed by decapitation. Brains were gently removed and chilled in a cold oxygenated buffer solution of the following composition: 119 mM NaCl, 4.6 mM KCl, 1.5 mM CaCl₂, 1.2 mM MgCl₂, 1.2 mM NaH₂PO₄, 15 mM NaHCO₃ and 5.5 mM Glucose; pH 7.4. The basilar artery (BA) was carefully dissected from the brain in a physiological buffer solution followed by either OC (naïve animals) or directly mounted in a wire myograph (arteries from rats which has undergone surgical procedures). Segments were incubated for 48 hours at 37° C. in humidified 5% CO₂/O₂ in DMEM supplemented with streptomycin and penicillin with inhibitors or vehicle (DMSO). Culture media was changed after 24 hours.

Myograph—Ex Vivo Pharmacology (OC and In Vivo)

For contractility measurements, both incubated BAs (ex vivo) and BAs after surgical procedure (in vivo) were cut into segments and mounted on a pair of metal wires (40 μm) in a myograph bath. The arteries from OC were mounted the same way after 48 hours in media. One wire was attached to a micro-meter screw which allows for fine adjustments of the distance between the wires, controlling the vascular tone. The second wire was connected to a force displacement transducer paired together with an analogue-digital converter (AD Instruments, Oxford, UK).

The segments were equilibrated in physiological buffer aerated with 95% O₂/5% CO₂, pH of 7.4, temperature set at 37° C. and the wires were separated for isometric pretension at 2 Nm⁻¹. The arterial segments were exposed two or three times with 60 mM K⁺, by exchanging the buffer with a 60 mM K⁺ buffer solution. To maintain equal osmolarity, a proportional amount of Na⁺ had been removed from the buffer. An absolute cut-off was set at 2.0 mN K⁺ₘₐₓ for inclusion of arterial segments from rats that underwent the surgical procedure. Endothelium function was evaluated with the addition of 5-HT (3.10⁻⁷ M) followed by carbachol (10⁻⁵ M). For arteries from the OC the protocol was as followed: first a cumulative concentration-response curve to sarafotoxin 6C (S6c, 10⁻¹⁴-10⁻⁷ M), followed by a concentration-response curve to endothelin-1 (ET-1, 10⁻¹⁴-10⁻⁷ M). At peak ET-1 (10⁻⁷ M), the buffer was changed to a Ca²⁺-free buffer containing 10⁻⁷ M ET-1 and nimodipine (L-type of voltage dependent Ca²⁺ channel entry blocker, 10⁻⁷ M). A concentration-response curve to Ca²⁺ (0.0125-3 mM) was then performed. When vasodilation was investigated arteries was precontracted with U46619 (1.10⁻⁷-3.10⁻⁷ M) or K⁺ (41 mM) and cumulative concentration-response curves was performed by adding calcitonin gene-related peptide (CGRP, 10⁻¹²-10⁻⁷ M), carbachol (10⁻¹⁰-10⁻⁵ M) or SNP (10⁻¹¹-10⁻⁴ M).

Two arterial segments from each operated animal were selected for either a cumulative concentration-response curve to ET-1 (10⁻¹⁴-10⁻⁷ M) or an ET-1 precontracted (10⁻⁷ M) Ca²⁺ concentration-response curve (0.0125-3 mM) in the presence of nimodipine (10⁻⁷ M). If there was only one curve above the cut-off, the concentration-response curve to ET-1 was prioritized. Segments with the highest K⁺ response were generally allocated to the concentration-response curve to ET-1. Concentration-response curves to Ca²⁺ were performed by adding increasing volumes of CaCl₂, from a 125 mM stock solution, to a Ca²⁺-free buffer solution. The Ca²⁺ free buffer solution had similar composition as above, but 1.5 mM CaCl₂ was exchanged with 0.03 mM EDTA.

Statistics and Data Acquisition

Contractile responses of each segment were adjusted according to the length of the artery and are expressed as mN/mm (Nm⁻¹). If the responses to 60 mM K⁺ was not significantly different, contractility data is shown as percentage of the individual vessel 60 mM K^{+max} plateau response from baseline. To compare ET-1 sensitivity, arteries were normalized to the percentage of the individual vessel ET-1ₘₐₓ. When the artery reached maximum contraction before the last concentration was added, the curves were constrained to the max contraction. The relative log IC₅₀/log EC₅₀ is the concentration corresponding to a response midway between the estimates of the lower and upper plateaus. The Eₘₐₓ is the maximal contraction in the concentration response curve and ET-1ₘₐₓ is the maximal contraction to ET-1. All quantitative data is presented as mean±standard error of the mean (SEM), unless otherwise stated.

K⁺ and endothelium-dependent responses were statistically compared by one-way ANOVA followed by Holm-Sidak's multiple comparison test (all groups compared to each other). Concentration-response curves were statistically compared by a two-way repeated measures ANOVA with Holm-Sidak's multiple comparison test and Geisser-Greenhouse correction for sphericity was used for the normalised ET-1ₘₐₓ data. A competitive curve fit was performed by comparing a biphasic vs. a non-linear regression curve fit, log (agonist)—variable slope. For all the ET-1 curves the biphasic regression model was accepted as the best curve fit. Significance of neurological assessment scores was evaluated with a two-tailed Fischer's exact test. Statistical analyses were done using Graphpad 8.02 software and significant p-values were defined as: *=p<0.05, **=p<0.01, ***=p<0.001.

Reagents

S6c was from PolyPeptide Group (Sweden), ET-1 was from Bachem (Germany) and CGRP from Tocris (UK). All MEK1/2 inhibitors except for U0126 were obtained from Selleckchem and dissolved in DMSO. U0126 monoethanolate (U120), DMSO (Sigma D2650) and all other chemicals were obtained from Sigma Aldrich.

Results

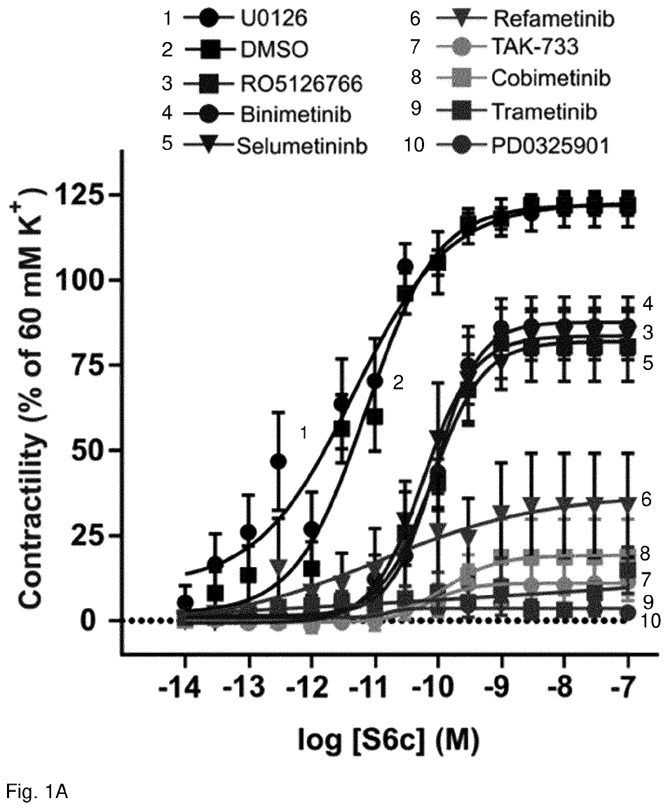

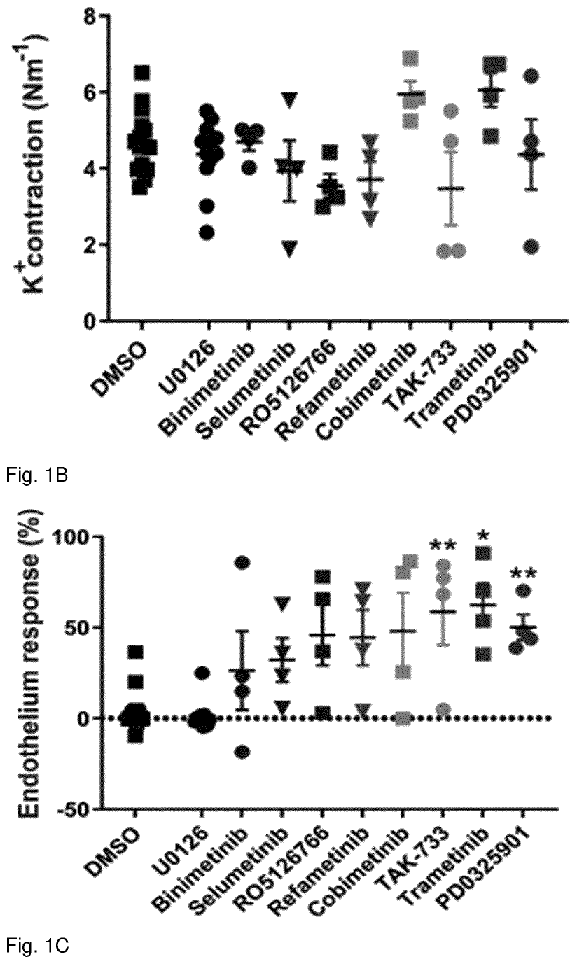

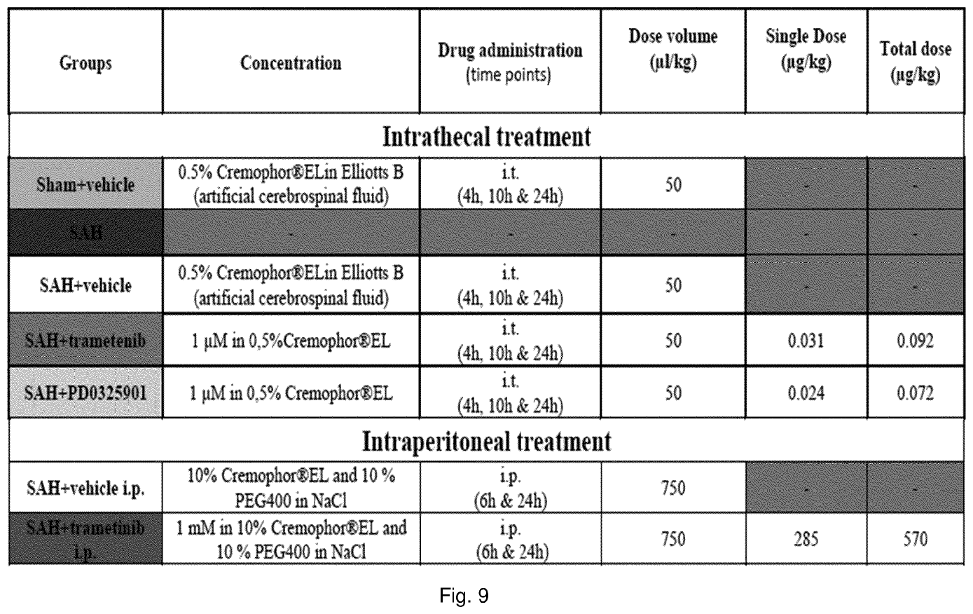

Organ culture (OC) was performed on rat basilar arteries (BAs) isolated from 588 rats (Sprague Dawley male rats, ˜320 g), and each BA was divided into 4 segments. A panel of MEK1/2 inhibitors was initially tested at a concentration of 1 μM, which is just below the established threshold for U0126 efficacy used till date (10 μM). FIG. 1A shows the contraction induced by the highly specific ET_{B} agonist, S6c, relative to the contraction induced by 60 mM K. Inhibitors were divided into 4 groups following the initial screening. The groups were as follows: I) Not effective at 1 μM vehicle (DMSO) and U0126. II) Some effect: Binimetinib, selumetinib and RO5126766. III) Borderline effective: Refametinib. IV) Highly effective: Cobimetinib, TAK-733, trametinib and PD0325901. The latter group was further divided in two groups based on the cell free IC₅₀ (FIG. 9), as the two subgroups could not be distinguished from each other at 1 μM.

There were no significant differences in the depolarization-induced contraction between arterial segments incubated with DMSO (vehicle) or the MEK1/2 inhibitors (FIG. 1B). The endothelial function of the arteries was also investigated. This was tested by applying 10-5 M carbachol to arteries precontracted with 3·10-7 M of 5-HT. OC with DMSO or U0126 were both associated with poor endothelial response to carbachol (FIG. 1C). Interestingly, there were significant differences between some of the groups, which appear to follow the same tendency as observed for the inhibition of S6c (FIG. 1A). Three of the MEK1/2 inhibitors, TAK-733 (P=0.0065), trametinib (P=0.0026) and PD0325901 (P=0.0474) had significant better endothelium function compared to the DMSO vehicle.

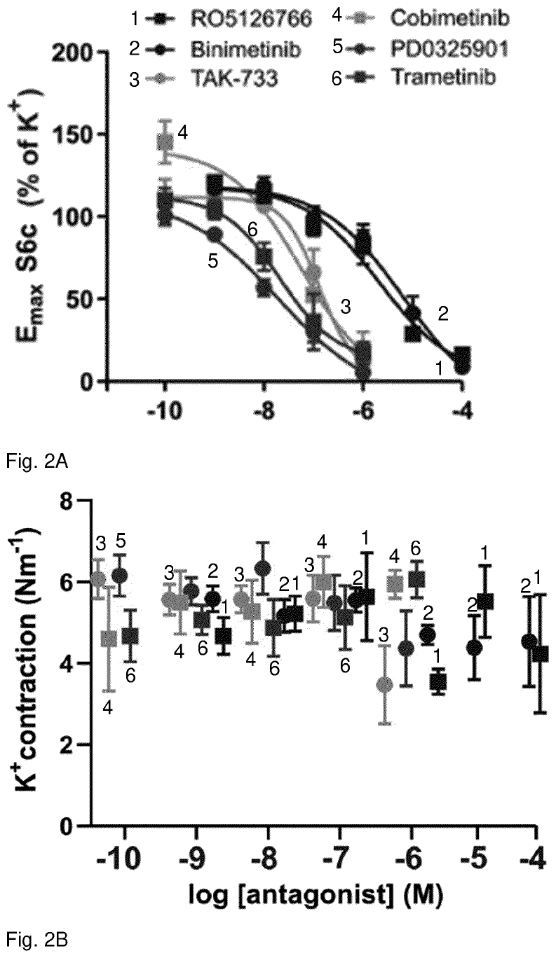

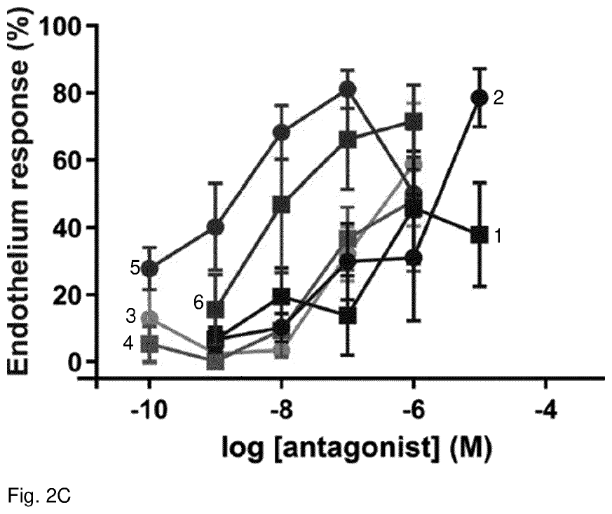

Selected inhibitors were characterized. The IC₅₀ values were determined for the six selected candidates (based on the Eₘₐₓ for S6c) using whole log concentrations ranging from no inhibition till near maximal inhibition of S6c-induced contraction (FIG. 2A). Binimetinib (pIC₅₀ 5.17±0.46) and RO5126766 (pIC₅₀ 5.68±0.26) were the least potent inhibitors which correlates with the data in FIG. 1. When analysing the concentration-response curves to the inhibitors, it is evident that TAK-733 (pIC₅₀ 6.89±0.31) and cobimetinib (pIC₅₀ 7.25±0.51) are less potent than trametinib (pIC₅₀ 7.68±0.32) and PD0325901 (pIC₅₀ 7.71±0.29) (FIG. 2A), which was not observed in FIG. 1.

Further, it was investigated if there were any effects on depolarization (60 mM K⁺) induced contractility of the arteries (FIG. 2B). There was no significant difference between the groups and no obvious concentration-dependent effect was observed. The initial screen (FIG. 1C) showed interesting effects of the MEK1/2 inhibitors on the endothelium function. FIG. 2C shows a concentration-dependent effect of the MEK1/2 inhibitors on the endothelium function in response to carbachol, with a similar trend for the inhibitors observed in the concentration-response curves for the Eₘₐₓ of S6c (FIG. 2A).

Conclusion

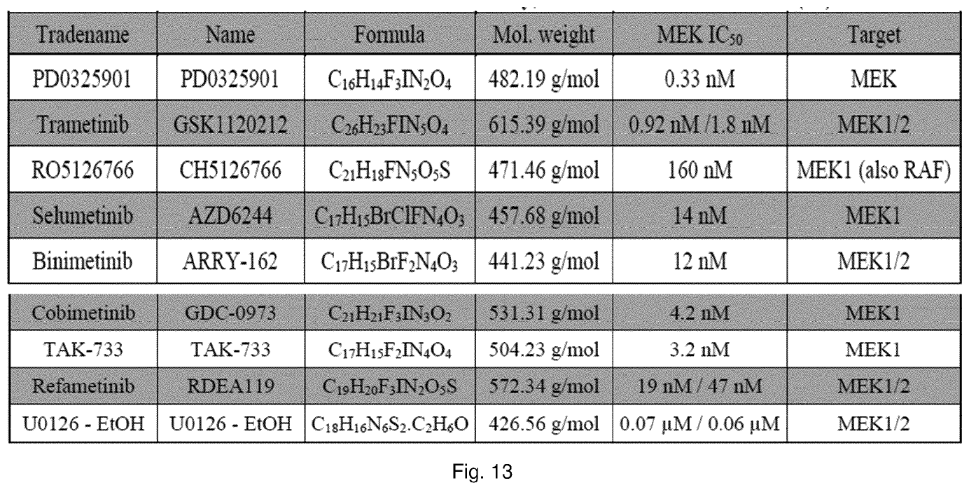

It is known that cerebral arteries treated with 10 μM U0126 in 48 hours OC shows an inhibitory effect on the ET_{B} specific S6c-induced contractility. In the present disclosure, 1 μM U0126 showed no significant effect, whereas trametinib and PD0325901 at 1 μM almost completely inhibited the contractile response after 48 hours organ culture (FIG. 1A). The data from the OC experiments illustrate a strong connection between MEK1/2 inhibition and functional upregulation of ET_{B} receptors, assessed with the specific ET_{B} agonist S6c. The cell free IC₅₀ values (FIG. 13) also correlate well with the IC50 values for the inhibition of the ET_{B} receptor upregulation (FIG. 2A). This support the link between MEK1/2 inhibition and functional receptor changes in the cerebral artery.

Hence, functional upregulation of ET_{B} receptors ex vivo can be completely prevented by inhibiting a signalling pathway that includes MEK1/2.

### Example 2

### Effect of Potent MEK1/2 Inhibitors on Pathways Regulating Vasomotion After 48 Hours Organ Culture of Basilar Artery

Materials and Methods

Methods were carried out as outlined in the previous example.

Results

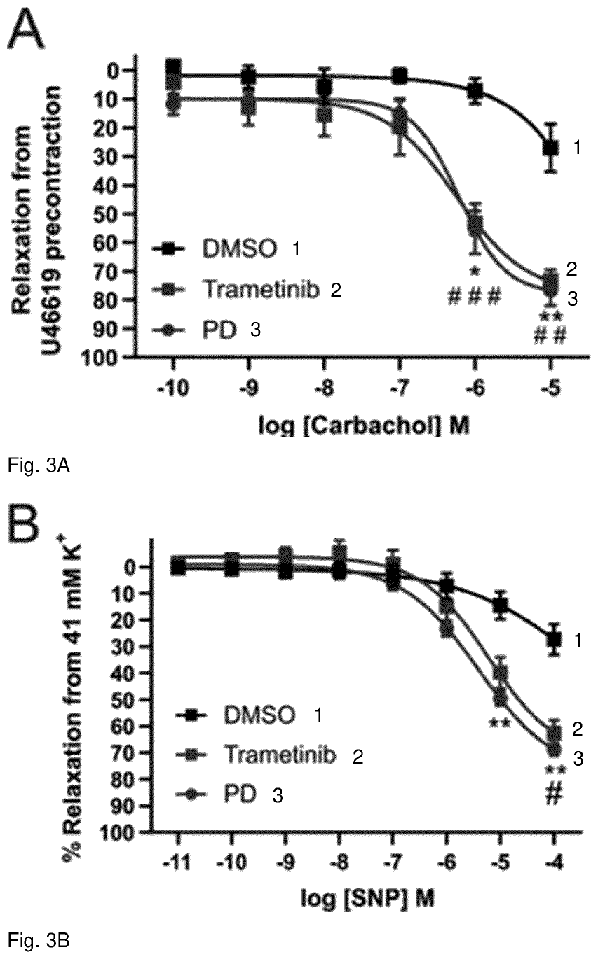

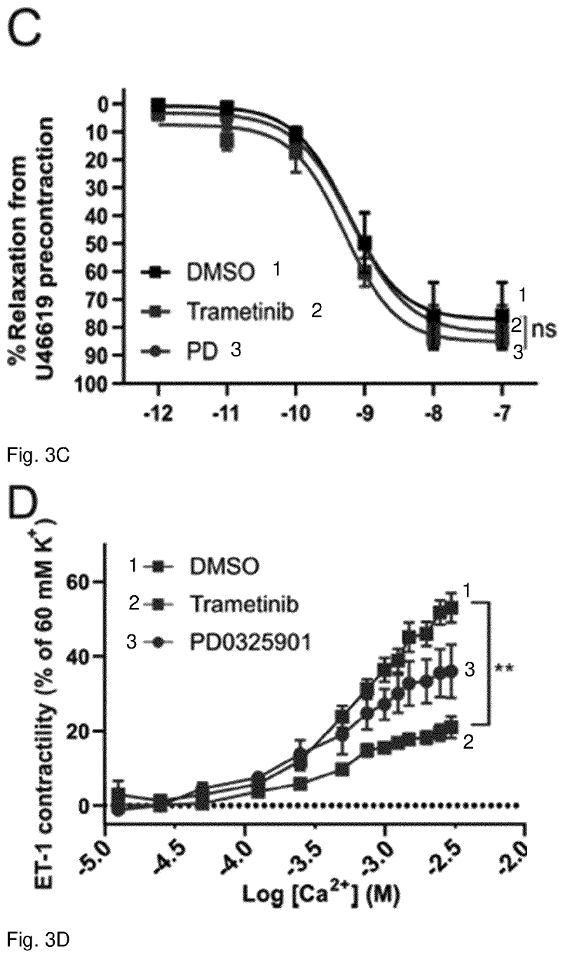

Since the arteries incubated with the most potent MEK1/2 inhibitors indicated preserved endothelium function, the possible cause of this improvement was further investigated. Arteries after OC with 1 μM of trametinib or PD0325901 were precontracted with the thromboxane A2 agonist, U46619 (1·10⁻⁷-3·10⁻⁷ M) or K⁺ (41 mM). There were no significant differences in the level of precontraction between the groups. In this new set of experiments, the improved vasodilation in response to carbachol was confirmed (10⁻¹⁰-10⁻⁵ M, FIG. 3A). This effect of carbachol could either be caused by improved endothelial NO release or changes in NO sensitivity in the vascular smooth muscle cell (VSMC). As shown in FIG. 3B there were significant differences in the Eₘₐₓ following the addition of the NO donor sodium nitroprusside (SNP, 10⁻¹¹-10⁻⁴ M). This indicates changes in the cGMP and NO sensitive signalling in the VSMCs, as the cause of the increased apparent endothelium function following incubation with MEK1/2 inhibitors. Since both cGMP and cAMP are important in regulating vasomotion in cerebral arteries, a signalling pathway that links to the generation of cAMP was also investigated. No differences in the response to CGRP (10⁻¹²-10⁻⁷ M, FIG. 3C) were observed.

It is known that the cerebral arteries exhibit a VDCC (voltage-dependent calcium channel) independent contraction after cerebral ischemia and SAH, which is not present in the fresh arteries. To further characterize changes in the cellular pathways leading to enhanced contraction, a concentration-response curve to Ca²⁺, by adding Ca²⁺ to BAs precontracted with ET-1 in Ca²⁺ free buffer containing 10⁻⁷ M nimodipine (L-type VDCC inhibitor) was performed. FIG. 3D shows the VDCC independent contraction of the BAs, where only trametinib (P=0.019) significantly prevented this increased VDCC independent contraction.

Conclusion

In non-SAH cerebral arteries or in fresh arteries the VDCC dominates, which can be blocked by nimodipine (standard treatment in SAH). In the present disclosure it is found that in SAH and in 48 h OC there is a change in the calcium channels. These procedures result in increased expression in VDCC of the independent type, i.e. cannot be blocked by the standard treatment with nimodipine. Trametinib was found to significantly prevent this increased VDCC independent contraction which (i) normalize the calcium channel expression, and (ii) likely makes the subject suitable for the therapy with nimodipine.

### Example 3

### Comparison of Trametinib and PD0325901 in Rats

Materials and Methods

Rat Subarachnoid Haemorrhage Model (In Vivo Model)

Rats were anesthetized and prepared for cisternal infusion of autologous blood, which simulates a SAH. Sham-operated rats went through the same procedure, omitting the intracisternal blood injection of 300 μL. At the end of the procedure, a PinPort (PNP3F22, Instech, US) was placed at the end of the ICP catheter to provide access for intrathecal (i.t.) injectable treatments by a PinPort injector (PNP-3M, Instech, US). 24 hours post-surgery, the rats received s.c. injections of Carprofen (Norodyl, 5 mg/kg) (Scanvet, Denmark) for analgesia.

In Vivo Treatment Regimens

The concentrations and doses of trametinib and PD0325901 used in this disclosure, is based on the ex vivo data from this present disclosure. All treatments were blinded throughout the study and all treatment regimens can be found in FIG. 9.

Intrathecal Treatment

The intrathecal (i.t.) treatment volumes were estimated for a cerebrospinal fluid (CSF) volume of approximately 90 μL (21) and the total dose was administered as three treatments (4 hours, 10 hours and 24 hours) through the PinPort in the ICP catheter placed in the cisterna magna during the surgical procedure. The first and third injections were given under fixation of the rat. Since the treatment 10 hours post-surgery was administered by a single researcher, the rats were briefly anaesthetised with isoflurane 3.5-4% (maintained at 1.75-2%) in atmospheric air/O₂ (70%/30%) using a facemask, to prevent sudden movements of the rat. Animals were given 2.5 ml isotonic saline s.c. to avoid dehydration, immediately after surgery and in conjunction with the 10 hours and 24 hours treatments. All in vivo treatments were dissolved in 0.5% cremophor EL (Kolliphor EL) in Elliott's B (artificial CSF): NaCl 125 mM, NaHCO₃ 23 mM, Dextrose 4 mM, MgSO₄ 1 mM, KCl 4 mM, CaCl₂, 1 mM and Na₂HPO₄ 1 mM.

Surgical Parameters—i.t. Groups

In all 41 rats, mean arterial blood pressure (MABP), pH, pCO₂, pO₂, ICP (138.4±6.9 mmHg) and temperature were within acceptable physiologic limits during surgery. There was one example of post-surgery mortality at 24 hours after the SAH in the SAH+vehicle group.

Results

Two most potent MEK1/2 inhibitors identified in the OC studies to a rat model of SAH were further investigated. Trametinib and PD0325901 had the highest potency, meaning that they potentially can be applied in smaller volumes than the current drug of choice, U0126. Following a 1 μM, 15 μL i.t. injections into CSF volume of the rat (assumed to be 90 μL), a CSF concentration of approximately 10⁻⁷ M after dilution was predicted. This corresponds to an estimated 75% inhibitor effect in the ex vivo OC study (FIG. 2A).

Conclusion

Trametinib was found to have high potency, indicating that it can be applied at lower doses than the current drug of choice, U0126. The first drug of choice U0126 was found to have similar advantageous effects in vivo intrathecally, but due to its poor solubility it was not possible to transform this agent to systemic administration. Trametinib was found to have excellent solubility and potency which demonstrates that it can be used in lower volumes, allowing for systemic use while still showing advantageous anti-SAH parameters.

### Example 4

### Effect of i.t. Trametinib and PD0325901 Treatment on Contractile Responses to 60 mM K⁺ and on the Endothelium Function

Materials and Methods

Methods were carried out as outlined in the previous examples.

Results

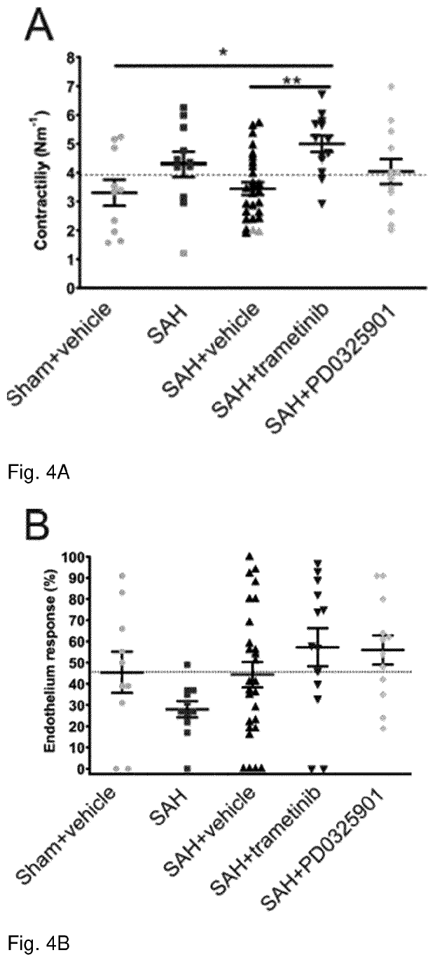

The contractile responses (Nm⁻¹) of all BAs to 60 mM K⁺ (including vessels below the cut-off) showed a significantly higher contractile response in the SAH+i.t. trametinib group (5.00±0.29 Nm⁻¹) compared to both the sham+i.t. vehicle group (3.30±0.45 Nm⁻¹) and SAH+i.t. vehicle group (3.45±0.22 Nm⁻¹) (FIG. 4A). The individual segment lengths (range 0.9—1.2 mm; 1.0±0.1 mm) were not significantly different between the groups. The SAH group had a slightly lower mean of the endothelium function compared to the other groups, but it was not significantly different (SAH vs. i.t. trametinib, P=0.1382) (FIG. 4B).

Conclusion

The use of MEK1/2 inhibitors with higher potency than U0126, allows for a concentration-dependent preservation of apparent endothelium function (FIG. 2C). A similar pattern was observed for both the endothelium function and on the functional upregulation of contractile ET_{B} receptors (FIG. 2A). Therefore, the MEK1/2 pathway appears to be involved in the disruption of endothelial and VSMC signalling in response to the reduction of blood flow through the artery. This contrasts with the neuronal vasodilation signalling, exemplified by CGRP, wherewith no change was observed (FIG. 3C). No significant changes in the endothelium function for the animals treated in vivo with neither trametinib nor PD0325901 due to relatively high variability was observed. Although both groups had higher mean values than the SAH group and SAH+i.t. vehicle group (FIG. 4B).

In contrast to the effect of trametinib or PD0325901 on ET_{B} receptor contractility and apparent endothelium function, no concentration-dependent effect of the inhibitors on the K⁺ responses after OC was observed (FIG. 2B). However, rats treated with i.t. trametinib (but not i.t. PD0325901), after experimental SAH showed higher K⁺ responses compared to the treatment with i.t. vehicle (FIG. 4A). Vessels incubated with i.t. trametinib or i.t. PD0325901 had K⁺ responses in the higher end of the compounds tested in the OC model (FIG. 1B).

### Example 5

### Effect of i.t. Trametinib and PD0325901 Treatment on Contractile Responses to ET-1

Materials and Methods

Methods were carried out as outlined in the previous examples.

Results

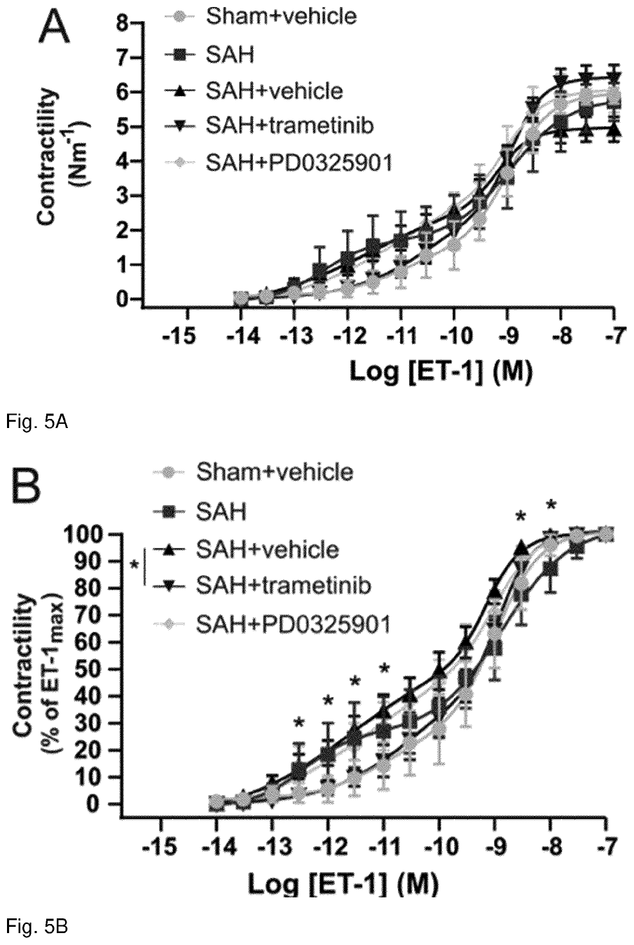

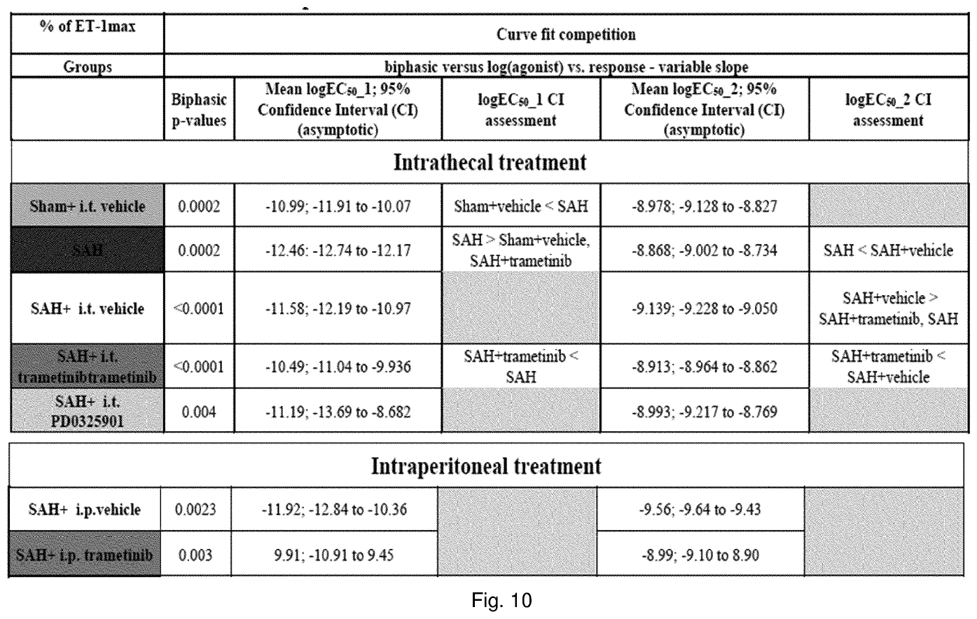

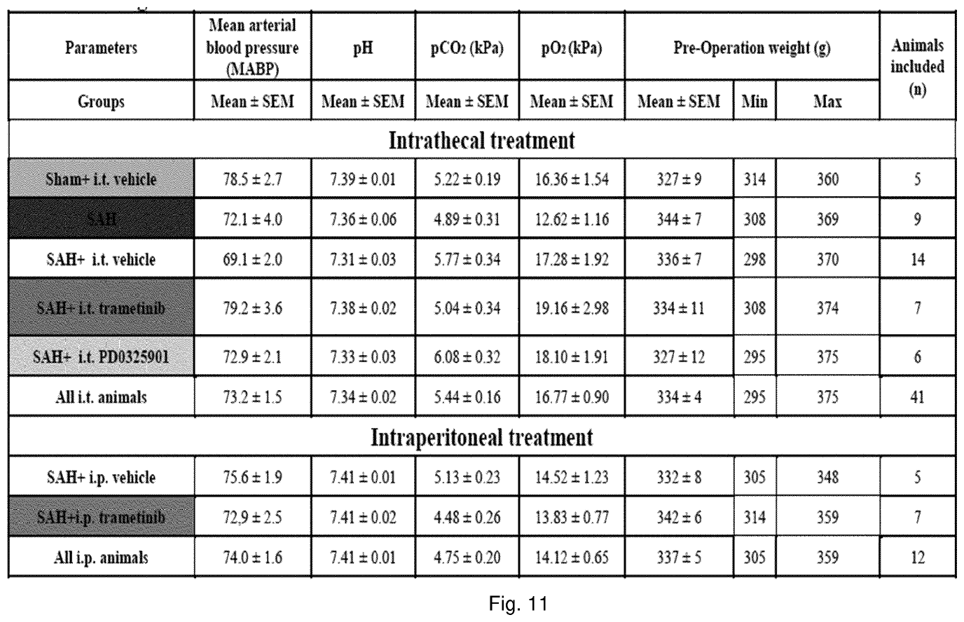

Since the K⁺ responses were different, non-normalized data were initially used. BAs from all treatment regimens (FIG. 9), were compared by cumulative concentration-response curves to ET-1 (10⁻¹⁴-10⁻⁷ M). No significant differences between curves were observed (FIG. 5A). The ET-1 sensitivity of the arteries was investigated by normalising the curves to their own ET-1ₘₐₓ. For the ET-1ₘₐₓ normalised data, the contraction at low ET-1 concentrations (10^{-12.5}-10^{-11.0} M) was significantly reduced in the SAH+i.t. trametinib group compared to the SAH+vehicle group (FIG. 5B). A competitive curve fit was performed by comparing a biphasic vs. a four-parameter variable slope regression. For all groups the biphasic regression model was accepted as the best curve fit (FIG. 10). The log EC_{50 (1)} 95% confidence intervals (CI) for the SAH group (−12.74 to −12.17) had significantly higher sensitivity compared to the sham+i.t. vehicle group (−11.91 to −10.07) and SAH+i.t. trametinib group (−11.04 to −9.936). At log EC_{50 (2)}, the SAH+i.t. trametinib group (−8.964 to −8.862) was significantly less sensitive than the SAH+i.t. vehicle group (−9.228 to −9.050). The log EC_{50 (1)} values and log EC_{50 (2)} values were the similar for both the absolute curves (Nm⁻¹) and the ET-1ₘₐₓ normalised curves (FIGS. 5A and 5B). All the log EC_{50 (1)} values, log EC_{50 (2)} values, and 95% CI values can be found in FIG. 10.

Conclusion

SAH results in increased sensitivity to ET-1 which is a hallmark of the disease. The SAH+i.t. trametinib was found to be significantly less sensitive than the SAH+i.t. vehicle group to ET-1. Thus, trametinib was able to effectively stop the detrimental upregulation of endothelin receptors after SAH.

### Example 6

### Effect of i.t. Trametinib and PD0325901 Treatment on VDCC Independent Calcium Ion Contraction

Materials and Methods

Methods were carried out as outlined in the previous examples.

Results

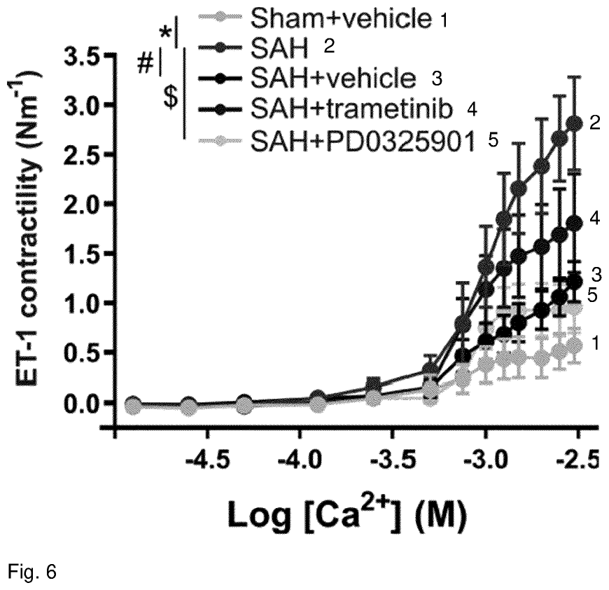

To investigate if the in vivo treatment affected the VDCCs independent contractility, cumulative concentration-response curves to Ca²⁺ (0.0125-3 mM) in ET-1 (10⁻⁷ M) pre-contracted BAs and in the presence of 10⁻⁷ M nimodipine were performed. The SAH group (2.8±0.5 Nm⁻¹) had a significantly higher nimodipine insensitive ET-1ₘₐₓ contraction compared to the sham+i.t. vehicle group (0.6±1.2 Nm-1), SAH+i.t. vehicle group (1.2±0.2 Nm⁻¹) and SAH+i.t. PD0325901 group (1.0±0.3 Nm⁻¹), while there was no significant difference between the SAH+i.t. trametinib group (1.8±0.5 Nm⁻¹) and the control groups (FIG. 6).

Conclusion

SAH resulted in a higher degree of nimodipine insensitive calcium channel responses. Trametinib treatment normalized the VDCC independent contraction to a level similar to that of the control.

### Example 7

### Neurological Assessment of i.t. Trametinib and PD0325901 Treatment Effects

Materials and Methods

Neurological Assessment—Rotating Pole Test

Gross sensorimotor function was evaluated using a rotating pole test, including a baseline evaluation on the day before induction of SAH. Briefly, movement across a 10-rpm rotating pole (45 mm diameter, 150 cm length) was evaluated with a cage at the end that contain the rat's own bedding material (“smells like home”). Rat performance was scored according to the following definitions: Low, the animal is unable to cross the pole without falling off; High, the animal can traverse the entire pole without falling off. All animals were trained to traverse the pole before surgery. Pre-SAH and on day 1 and 2 after surgery, each animal was scored twice for left and right rotation respectively, i.e. 4 counts per animal. Animals were graded by personnel blinded to the experimental groups of the animals. Data are shown percentage as % high score count/total score count.

Results

The rotating pole test performed herein is not a pure motor function test, as it does require training of the rats in advance. In addition to the learning aspect, the fact that the pole is rotating also leads to motivation being a factor of success. Therefore memory, motivation and attention are involved in a successful score. The rats were scored at three time points: Pre-SAH, 24 hours and 48 hours post-SAH (Data are shown as % high score count/total score count).

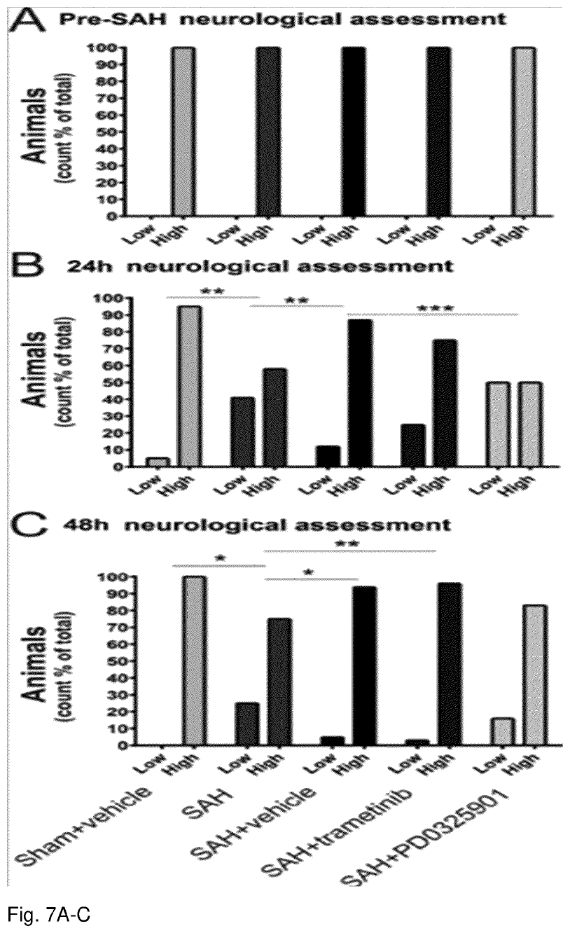

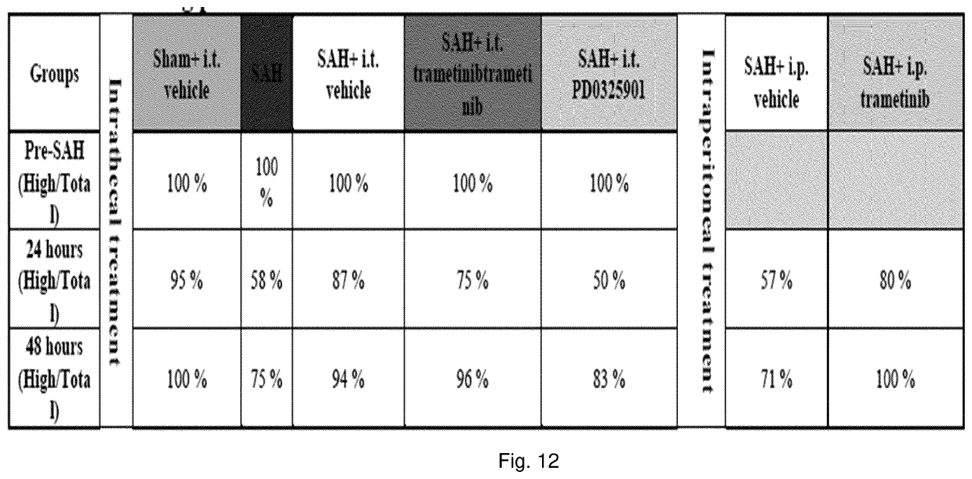

All rats scored 100% in the rotating pole test prior the surgery (FIG. 7A). Neurological score deficits was seen for all groups comparing 24 hours post-SAH with pre-SAH, except for the sham+i.t. vehicle group (FIG. 7A-C, FIG. 12). Rats after experimental SAH demonstrated significantly worsened neurological score after 48 hours, when compared to pre-SAH (Scorepre 100% to Score48 h 75%, P=0.0022). At the 48 hours endpoint the rats in the SAH+i.t. trametinib (Score48 h 96%), sham+i.t. vehicle (Score48 h 100%) and SAH+i.t. vehicle (Score48 h 94%) groups had a significantly higher neurological score than the rats in the SAH group (Score48 h 75%) (FIG. 7C). See FIG. 12 for all score percentages.

Conclusion

Improved neurological assessment scores (rotating pole test) were observed for trametinib, but not with the pharmacokinetically unstable PD0325901, which supports the involvement of the MEK1/2 pathway in the phenotypical modulation observed in VSMCs following SAH.

### Example 8

### Effect of i.p. Trametinib Treatment on Contractile Responses to 60 mM K⁺, ET-1 and Neurological Assessment

Materials and Methods

Intraperitoneal Treatment

For the intraperitoneal treatment (i.p) the compound was dissolved in 10% cremophor EL (Kolliphor EL) and 10% PEG400 in NaCl, which also served as vehicle. The total dose was administered as two treatments (6 hours and 24 hours). Animals were given 2.5 ml isotonic saline s.c. to avoid dehydration, immediately after surgery and in conjunction with the treatments.

Surgical Parameters—i. p. Groups

In all 11 rats, mean arterial blood pressure (MABP), pH, pCO₂, pO₂, ICP (124.2±6.3 mmHg) and temperature were within acceptable physiologic limits during surgery.

Results

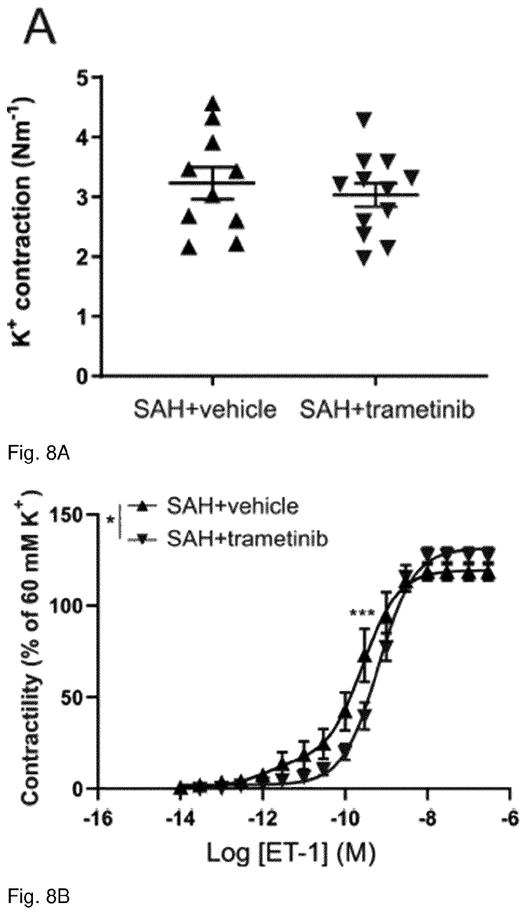

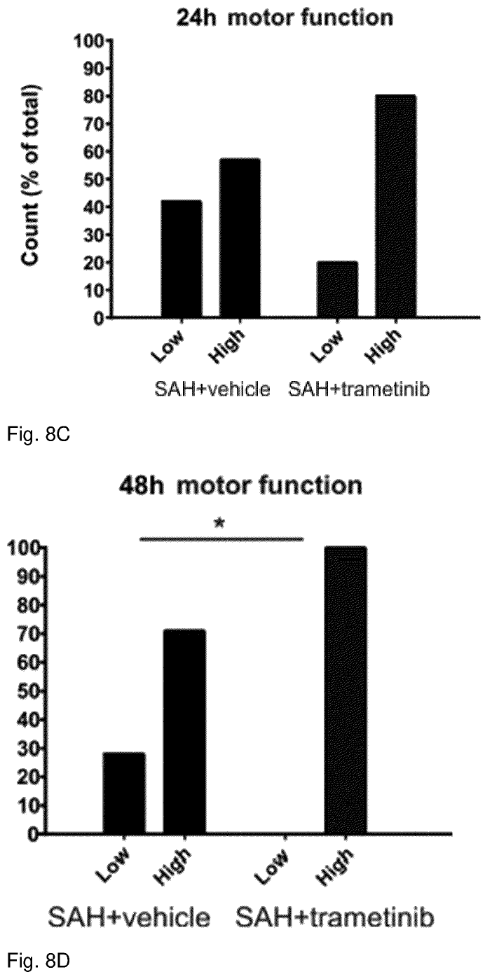

Going further from the i.t. proof of concept study, trametinib was tested using an i.p. injection treatment protocol. Animals were exposed to SAH and treated with i.p. injections of trametinib or vehicle at 6 and 24 hours post SAH. 48 hours after induced experimental SAH, arteries were isolated for the wire myograph. The individual segment lengths (range 0.9—1.2 mm; 1.13±0.02 mm) were not significantly different between the SAH+i.p. vehicle or SAH+i.p. trametinib. The contractile responses (Nm⁻¹) of all BAs to 60 mM K⁺ did not show any difference when comparing the SAH+i.p. trametinib group (3.03±0.19 Nm-1) with the SAH+i.p. vehicle group (3.23±0.27 Nm⁻¹) (FIG. 8A). This contrasts with the SAH+i.t. treatment (FIG. 4A).

The SAH+i.p. trametinib group and SAH+i.p. vehicle groups were compared by cumulative concentration-response curves to ET-1 (10⁻¹⁴-10⁻⁷ M). There was a significant decrease in contractility (at 10^{-9.5} M) for the SAH+i.p. trametinib group compared to the SAH+i.p. vehicle group. A competitive curve fit was performed by comparing a biphasic vs. a four-parameter variable slope regression. For both groups the biphasic regression model was accepted as the best curve fit, and the log EC_{50 (1)} values, log EC_{50 (2)} values and 95% CI values can be found in FIG. 10. The same animals were evaluated for neurological deficits by the rotating pole test (FIGS. 8C/D). The rats were scored at two time points: 24 hours and 48 hours post-SAH (Data are shown as % high score count/total score count). At the 48 hours endpoint the rats in the SAH+i.p. trametinib (Score48 h 100%), scored significantly better (p=0.0143) than SAH+i.p. vehicle (Score48 h 71%). See FIG. 12 for all score percentages.

Conclusion

A significant decrease in contractility for the SAH+i.p. trametinib rats was found compared to the SAH+i.p. vehicle group. Rats in the SAH+i.p. trametinib scored significantly better than SAH+i.p. vehicle in a rotating pole test.

### Example 9

### Effect of Subacute Phase in of Subarachnoid Haemorrhage in Female Rats

Materials and Methods

Animals

Female Sprague-Dawley rats (NTac:SD, Taconic Denmark), were kept at a constant temperature (22±2° C.) and humidity (55±10%) with a daily rhythm of 12-hour light/12-hour dark, provided with standard chow (Altromin, Scanbur, Denmark) and water ad libutum. Rats were generally housed in Eurostandard cages (Type VI with 123-Lid) 2-6 together and single housed (Type III with 123-Lid) after the surgical procedure. All rats were acclimatized for 5-7 weeks before experiments.

Vaginal Smears—Estrous Cycle Determination

Two weeks prior to SAH surgery, the estrous cycle of each rat was monitored daily by collection of vaginal smears for microscopical characterization of the types of cells present. In order to minimize potential experimental variability due to fluctuations in estrogen levels, female rats in proestrus were excluded from the study.

Experimental Model of SAH

All procedures were performed strictly within national laws and guidelines and were approved by the Danish Animal Experimentation Inspectorate (License no. 2016-15-0201-00940).

SAH was induced as for male rats. Female Sprague-Dawley rats (230-300 g, 14-17 weeks) were anesthetised by subcutaneous (s.c) administration of either a mixture of hypnorm/midazolam (0.25 ml/kg of a mixture of Hypnorm (fentanyl citrate (0.16 mg/kg), fluanison (5.0 mg/kg) and midazolam (Hameln Pharma, Germany) (2.0 mg/kg)) or intraperitoneal (i.p) administration of a mixture of ketamin/xylazine (1.5 ml/kg of a 3:2 mixture of Ketamin (MSD Animal Health) (100 mg/ml) and Xylazine (KVP pharma, Germany) (20 mg/ml)) and thereafter intubated and ventilated with 30% O₂ and 70% atmospheric air. Blood samples were regularly analysed (PaO₂, PaCO₂ and pH) in a blood gas analyser (ABL80 FLEX, Radiometer, Denmark). Body temperature was kept at 37° C.±0.5° C. with a regulated heating pad (TC-1000, CWE, Inc., PA, USA). MABP and ICP were continuously measured via catheters inserted into the tail artery and the cisterna magna, respectively, connected to pressure transducers and a Powerlab unit and recorded by the LabChart software (both from AD Instruments, Oxford, UK). A laser-Doppler blood flow meter probe (Oxford Optronix, UK) was placed on the dura mater through a hole in the skull drilled 4 mm anterior from bregma and 3 mm rightwards of the midline (regularly chilled by saline irrigation during the procedure). Through a second hole drilled 6.5 mm anterior to bregma in the midline, a 25G Spinocan® cannula (REF:4505905, B. Braun Melsungen AG, Germany) was descended stereotactically at an angle of 30° to the vertical plane towards a final position of the tip immediate anteriorly to the chiasma opticum. After 10 minutes of equilibration, 250 μL or 300 μL of blood was withdrawn from the tail catheter and injected manually through the cannula. The pressure and rate of the blood injections were manually controlled aiming at raising ICP to the higher range of mean MABP levels in all animals (app. 150 mmHg) and at the same time, the injection rate was controlled to produce an acute and prolonged drop in CBF. Subsequently, rats were maintained under anaesthesia for another 30 minutes. At the end of the procedure, the tip of the ICP catheter was sealed with a PinPort (PNP3F22, Instech, US), for later measurements of the ICP and the tail catheter, needle and laser-Doppler probe were carefully removed, and incisions closed. Rats were thereafter revitalised and extubated. At the end of surgery and once daily thereafter, rats received subcutaneous injections of Carprofen (5 mg/kg, Scan Vet, Denmark) and 2.5 mL isotonic saline. Sham-operated rats went through the same procedure with the exception that no cannula was descended, and no blood was injected into the chiasma opticum. Rats were maintained in single cages until euthanasia by decapitation 2 days post-surgery.

Experimental Groups

In a preliminary study, SAH was induced in female rats by injecting 250 μL of autologous blood in the prechiasmatic cistern and the contractile responses of basilar arteries (BAs) and middle cerebral arteries (MCAs) were evaluated by myography (n=12, 7 SAH and 5 sham-operated). In the experiments for the actual study the female rat was injected with 300 μL of autologous blood. Sham-operated rats served as controls (n=34, 18 SAH and 16 sham-operated). The rats were divided randomly into either the SAH or sham group. A total number of 46 rats were utilized in the study.

Neurological Tests

a) Rotating Pole Test

Gross sensorimotor function was evaluated using a rotating pole test at different speeds (3 or 10 rpm). At one end of the pole (45 mm in diameter and 150 cm in length) a cage is placed with an entrance hole facing the pole. The floor of the cage is covered with bedding material from the home cage of the rat being tested. Rat performance was scored according to: Score 1, the animal is unable to balance on the pole and falls off immediately; Score 2, the animal balances on the pole but has severe difficulty crossing the pole and moves <30 cm; Score 3, the animal embraces the pole with its paws and does not reach the end of the pole but does manage to move >30 cm; Score 4, the animal traverses the pole but embraces the pole with its paws and/or jumps with its hind legs; Score 5, the animal traverses the pole with normal posture but with >3 foot slips; and Score 6, the animal traverses the pole perfectly with <3 foot slips. Before surgery all animals are trained until they achieved a Score 5 or 6. On Days 1 and 2 after surgery, each animal is tested twice on the static pole and 4 times at each rotation speed, twice with rotation to the left and twice with rotation to the right.

b) Behavioral Observation

Rats were observed once a day and their behaviour was scored according to the following parameters was assembled: body-temperature, body-posture (low or curved back), eyes (closed, dry or blood), fur (dirty or piloerection), faeces (dry or none), rat noises (when handling the rat), noise sensitive (hyperactive), temperament (passive, aggressive), movement, balance and ears (white). All 11 observations were scored as follows: normal state=0, medium state=1 and poor state=2. A mean score for each group was calculated for each day of observation.

Harvest of Cerebral Arteries

Rats were decapitated under CO₂ sedation two days after undergoing surgery. Brains were removed quickly and chilled in cold bicarbonate buffer solution. Basilar (BA) and middle cerebral (MCA) arteries were carefully dissected from the brain. For contractility measurements, the BAs and MCAs were cut into 1-1.5 mm-long cylindrical segments and mounted in a wire myograph.

In Vitro Pharmacology

For measurements of contractile responses of cerebral arteries, a myograph (Danish Myograph Technology A/S, Denmark) was used to record the isometric tension in segments of isolated arteries. Vessel segments were mounted on two 40 μm-diameter stainless steel wires in a wire myograph setup. The segments were then immersed in a temperature-controlled bicarbonate buffer solution (37° C.) of the following composition (mmol/L): NaCl 119, NaHCO₃ 15, KCl 4.6, MgCl₂ 1.2, NaH₂PO₄ 1.2, CaCl₂ 1.5, and glucose 5.5. The buffer is continuously aerated with 5% CO₂ in O₂, maintaining a pH of 7.4. Vessel segments were stretched to an optimal pretension (2mN) in a three-step process as previously found optimal and are then allowed to equilibrate at this tension for approximately 20-30 minutes. The vessels were then exposed to a bicarbonate buffer solution with 60 mM K⁺ obtained by partial substitution of 59.5 mmol/L NaCl for KCl in the above-described isotonic bicarbonate buffer solution. K⁺-evoked contractile responses were used as reference values for normalization of agonist-induced responses and to evaluate the depolarization induced contractile capacity of the vessels. Only BAs and MCAs with K⁺-induced responses >2 mN and >0.8 mN, respectively, were used for further evaluation. The presence of functional endothelium in the vessel segments was assessed by precontraction with 5-hydroxytryptamine (5-HT) (Sigma-Aldrich, H9523) (3×10⁻⁷ M) followed by relaxation with carbachol (Sigma-Aldrich, C4382) (10⁻⁵ M). A relaxant response to the cholinergic receptor agonist carbachol is to be considered indicative of a functional endothelium. Concentration-response curves were obtained by the cumulative application of the natural ligand for the endothelin receptors (ET_{A}/ET_{B}), ET-1 (Bachem, 4040254), in the concentration range of 10⁻¹⁴ to 10⁻⁷ M. Likewise, concentration-response curves to 5-carboxamidotryptamine (5-CT) (Sigma-Aldrich, C117), a 5-HT_{1B}/5-HT_{1D} agonist, were obtained by the cumulative application of 5-CT in the concentration range of 10⁻¹² to 10⁻⁵ M.

Intracranial Pressure Measurements

ICP recordings were performed 1- and 2-days post-surgery using a novel fluid-filled sealed off PinPort system developed for consecutive, real time ICP measurements in rats. The sealed PinPort (PNP3F22, Instech, US) of the cisterna magna catheter was connected to a pressure transducer via a fluid-filled tubing with a PinPort injector (PNP-3M, Instech, US). The pressure transducer was connected to a power lab and the ICP recorded by the LabChart software (AD Instruments, Oxford, UK). To get measurements undisturbed by movements, rats were sedated with 0.5 mL/kg midazolam (2.0 mg/kg) 15 minutes prior to ICP recording followed by recording of the ICP for 15 minutes, then the PinPort injector was removed and the rat returned to the animal facility.

Assessment of Brain Edema

After decapitation, the brain was quickly removed and placed in ice-cold bicarbonate buffer solution. The brain was divided into two intact cerebral hemispheres without the cerebellum. Each hemisphere was further divided into the following regions; striatum, hippocampus and cortex for regional determination of brain edema formation. Brain edema was assessed by comparing wet-to-dry ratios (WDR). Tissues were weighed (wet weight (ww)) with a scale to within 0.1 mg. Dry weight (dw) of the brain was measured after heating the tissue for 24 hours at 110° C. in a drying oven. Tissue water content was then calculated as % of water content in the brain with the following formula: (ww−dw/ww)×100%.

Statistics and Data Analysis

Data are expressed as mean±standard error of the mean (SEM), and n refers to the number of rats. For in vitro pharmacology studies, contractile responses are expressed as a percentage of the maximum 60 mM K⁺-induced contraction from baseline. Eₘₐₓ value represents the maximum contractile response elicited by an agonist and the pEC₅₀ is the negative logarithm of the drug concentration that elicited half the maximum response. For biphasic responses, Eₘₐₓ₁ and pEC_{50 1} describe the high-affinity phase and Eₘₐₓ₂ and pEC_{50 2} describe the low-affinity phase. Statistical differences between concentration-response curves, rotating pole and ICP measurements were analysed using two-way ANOVA with the Bonferroni post-test. When comparing two different observations/time points, the statistical difference was investigated using the unpaired t-test. Graphpad 5.1 software was used for statistical analyses and data presentation. Significant p-values were defined as: *: P=(<0.05), **: P=(<0.01), ***: P=(<0.001).

Results

Surgery, Vaginal Smears and Physiological Parameters

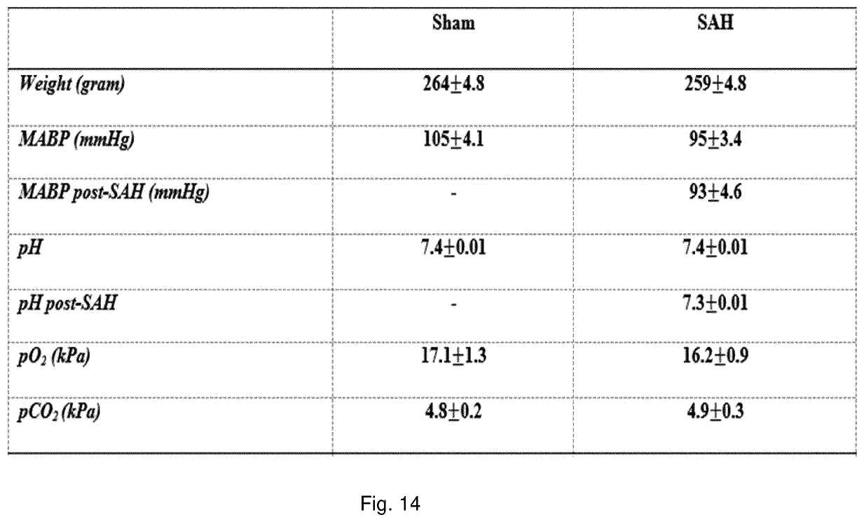

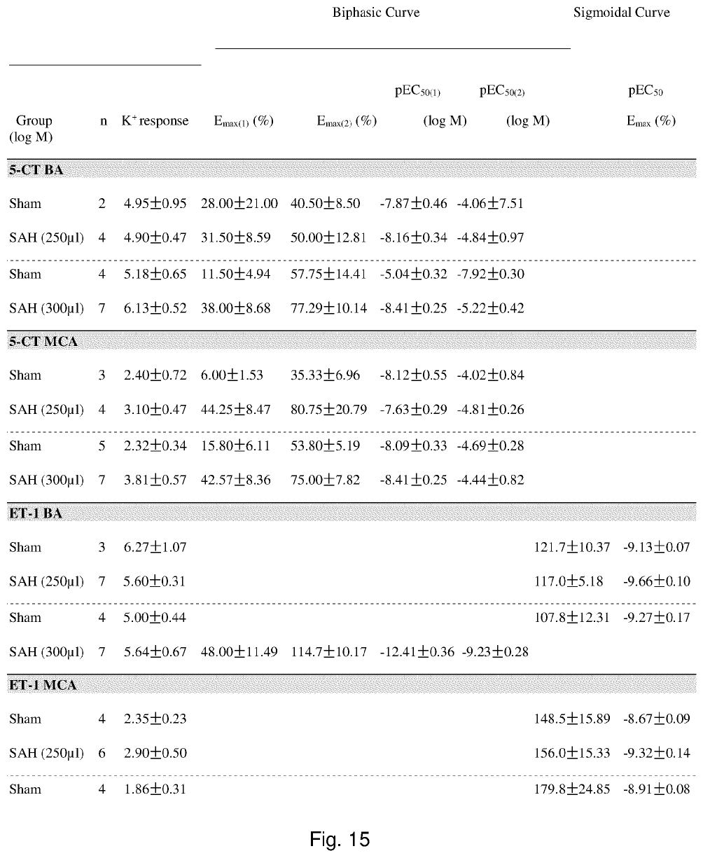

All rats survived the study. Two rats were excluded from the study due to their hormonal status (rats in proestrus with high levels of estrogen) on the day of surgery. The daily evaluation of vaginal smears confirmed that, on the day of surgery, the remaining rats were randomly divided relative to stage of the cycle (metestrus, diestrus, estrus) between the sham and SAH groups. There were no significant differences in physiological parameters (weight, MABP, pH, pO₂ or pCO₂) comparing SAH and sham-operated rats in neither the preliminary study (250 μL autologous blood injected, data not shown) or the main study (300 μL blood injected, FIG. 14). As a result of the blood injection, the cortical blood flow dropped to 16.24±6% of resting flow in female rats receiving 300 μL of autologous blood (FIG. 15).

Preliminary Study (Female Rats Receiving 250 μL Blood)

Partial Increased Cerebrovascular Constriction 2 Days After SAH in Female Rats Receiving 250 μL Autologous Blood Compared to Sham

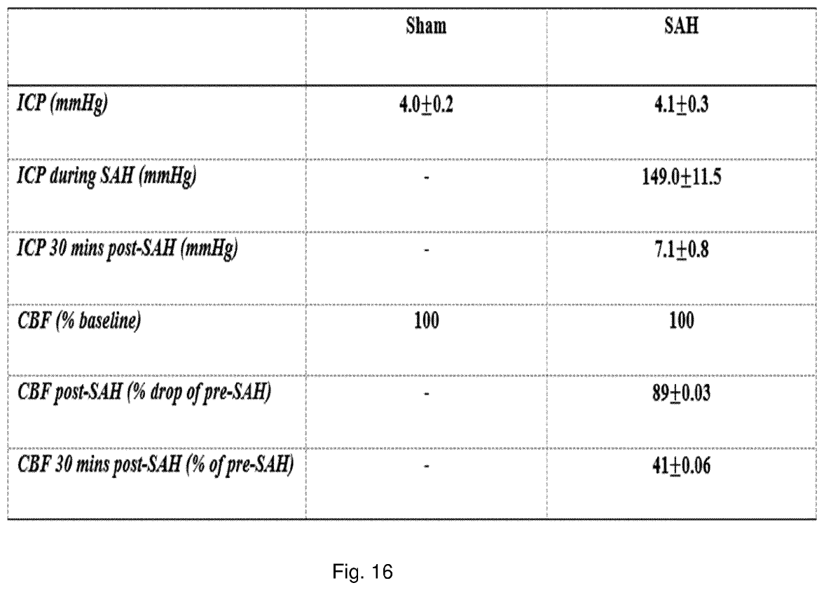

There were no differences in depolarization-induced constriction with 60 mM K⁺ enriched buffer comparing arterial segments (BA and MCA) from sham- and SAH-operated rats (FIG. 16). Likewise, there was no difference in endothelial mediated dilation studied by acetylcholine induced relaxation in 5-HT precontracted vessels comparing arterial segments (BA and MCA) from sham and SAH. It has previously been shown that arterial segments from male SAH-induced rats resulted in a left-ward shift of the ET-1 concentration-contraction curves with a transition into biphasic curves, whereas the ET-1 concentration-contraction curves for sham-operated male rats are sigmoidal. Biphasic curves after SAH in male rats reflects the occurrence of contractile ET_{B} receptors in the VSMC in addition to contractile ET_{A} receptors already present. In the preliminary study with SAH induction by injecting 250 μl autologous blood in female rats, ET-1 induced sigmoidal curves in both BA and MCA segments from SAH-induced rats and sham-operated rats, respectively. However, BAs and MCAs from female rats subjected to SAH resulted in a left-ward shift and a significantly increased sensitivity to ET-1 compared to sham (FIG. 16). In MCA segments from female SAH rats a significantly left-ward shift of 5-CT concentration-contraction curves compared to sham was observed, thus, in BAs there was no clear difference in 5-CT induced contraction between SAH and sham rats (FIG. 16).

Main Study (Female Rats Receiving 300 μL Blood)

The preliminary study showed that female rats subjected to SAH, receiving 250 μL blood intracisternally, induced increased vasoconstriction towards ET-1 and 5-CT in cerebral arteries. However, due to the lack of transition into biphasic curves in ET-1 induced concentration-response curves, the high variability in the data and no differences in 5-CT induced contraction in BA when comparing sham and SAH, the volume of blood injected was increased resulting in greater SAH-induced damage. Therefore, in the following results presented (main study), all rats subjected to SAH received 300 μL autologous blood intracisternally.

Reduced General Wellbeing and Sensorimotor Cognition After SAH in Female Rats

To assess whether SAH induce neurological deficits in female rats two different tests were used, observation of rat general wellbeing and a rotating pole test. As shown in FIG. 1a, SAH resulted in a significant decrease in the score of general wellbeing on both day 1 and 2 compared to sham. Furthermore, female rats subjected to SAH showed significantly impaired balance and movements when they transversed the wooden pole, whether with no rotation or at two different speeds of rotation. The sensorimotor deficits were significant at 1 and 2 days after SAH in female rats compared to sham.

Elevated Intracranial Pressure in the Subacute Phase After SAH in Female Rats Compared to Sham

The intracranial pressure was 4.0±0.2 mmHg in sham-operated rats at the day of surgery and the ICP did not change significantly 1 or 2 days after sham operation (FIG. 15). In experimental rats before SAH, the ICP was 4.1±0.3 mmHg. ICP was then increased transiently to an average of 149±11.5 mmHg at SAH induction and was 7.1±0.8 mmHg at 30 minutes after SAH (FIG. 15). The mean ICP of female rats was significantly increased on both day 1 and 2 after SAH compared to sham.

In female SAH rats, the ICP increased day 1 after surgery in all rats compared to pre-SAH levels, whereas at day 2, ICP either increased further (4/9 rats) or decreased (5/9 rats) in relation to day 1 levels. However, in the rats were the ICP decreased day 2 after SAH, the ICP was still increased compared to the ICP recorded pre-SAH in all 5/9 rats except for 1. In female sham-operated rats, the ICP increased slightly (5/9 rats) or remained at the pre-surgery level on day 1 after surgery and then on day 2, the ICP either decreased slightly (2/9), remained at pre-SAH level (3/9) or increased slightly (4/9) as compared to the ICP on day 1.

Increased Cerebrovascular Constriction 2 Days After SAH Compared to Sham in Female Rats

Potassium-induced responses with 60 mM K⁺ enriched buffer did not differ significantly between arterial segments (BA and MCA) from sham and SAH animals (FIG. 16). Furthermore, there was no difference in endothelial-mediated dilation comparing arterial segments (BA and MCA) from sham- and SAH-operated rats.

The concentration-contraction curve to ET-1 of BA segments from female SAH rats was shifted to the left with a beginning transition into a biphasic curve. BA segments from female SAH rats had significantly increased sensitivity to ET-1 compared to BA segments from sham-operated rats (FIG. 16). MCAs from SAH-induced female rats also showed a significantly elevated contraction to ET-1 compared to sham with biphasic curves (increased Eₘₐₓ₁). In contrast to ET-1 curves for BA segments, the MCA curves were not leftward shifted after SAH compared to sham (FIG. 16).

In male rats it has been demonstrated that cerebral arteries have significantly increased sensitivity to 5-CT after SAH compared to sham. These curves were shown to be leftward shifted which reflects upregulation of 5-HT_{1B} receptors. In both BA and MCA segments from female SAH rats there was a significantly increased sensitivity to 5-CT-induced contractions compared to sham. Concentration-response curves obtained for cerebral segments from female rats 2 days after SAH showed a leftward shifted compared to sham (FIG. 16).

Increased Brain Water Content 2 Days After SAH in Female Rats Compared to Sham

Edema formation was evaluated in the striatum, cortex and hippocampus separately, by calculating the brain water content in the isolated brain regions. There was significant increase in percentage of brain water in the cortex 2 days after SAH in female rats compared to sham (p<0.0473). The percentage of brain water tended to be increase in the hippocampus 2 days after SAH in female rats compared to sham (P=0.05). The brain water content in the striatum did not differ between SAH and sham-operated female rats (p<0.2711). These results point to localized edema in the cortex and in the hippocampus 2 days after SAH in female rats compared to sham.

Conclusion

SAH in female rats was shown to resemble the neurological damage seen in male rats after SAH with decreased general wellbeing and significantly decreased sensorimotor function. A significant increase in ICP on both day 1 and 2 after SAH in female rats was demonstrated. The course of changes in ICP over the first days after SAH may allow prediction of EBI and DCI severity. SAH in female rats resulted in increased vascular contractility to ET-1 and 5-CT in cerebral arteries. Targeting vascular changes in order to prevent delayed neurological damage after SAH is thus as a therapeutic strategy in both males and females.

Hence, prevention of these gender-independent mechanisms provides the basis for a universal treatment strategy for DCI after SAH.

### Example 10

### Effect of Ovariectomy on Vasomotor Responses of Rat Middle Cerebral Arteries After Focal Cerebral Ischemia in Female Rats

Materials and Methods

Ethics

The study design was approved by Lund County Administrative Court (M178-11, M8-09). All procedures and animal treatments followed the guidelines of the Ethics Committee of Lund University. The study complies with the ARRIVE guidelines (Animals Research: Reporting in Vivo Experiments).

In Vivo Hormone Treatment

The rats were ovariectomized by the vendor (Charles River, L'Arbresle Cedex, France) and treated with subcutaneously implanted silastic capsules (1.57 mm ID×3.18 mm OD, Dow Corning, Hemlock, Mich., USA) that contained either progesterone (9 mm length) or 17β-estradiol (5 mm length) to restore hormone levels. Empty silastic capsules of appropriate lengths were used as placebo. The ovariectomy and implantation of capsules were performed during the same session. This protocol has been shown to produce levels of 17β-estradiol and progesterone within the physiological range.

Dry uterine weight and serum 17β-estradiol were measured at the time of euthanasia to verify effective estrogen replacement. Trunk blood or blood from cardiac puncture was collected at the time of euthanasia in plain tubes and left to coagulate at room temperature for 40 min followed by centrifugation at 2,000×g for 12 min at 4° C. The supernatant was collected and stored in aliquots at −80° C. until time of determination of 17β-estradiol (radioimmunoassay). The detection limit of 17β-estradiol in the radioimmunoassay was 11 pg/mL.

After 3 weeks of hormone treatment, the estrogen treated ovariectomized rats (OVX+E) had, in comparison with ovariectomized (OVX) animals, a lower body weight (187±4 g vs. 254±8 g, P<0.05) and higher uterine weight (98±16 g vs. 19±1 g, P<0.05). The serum levels of 17β-estradiol in the OVX+E animals were within the physiological range (26±2 pg/mL), whereas the level in the ovariectomized animals was below the detection limit (<11 pg/mL in all samples; p<0.05).

Female rats with intact ovaries were included as controls (hereafter referred to as “intact”). The estrous cycle in the intact animals was monitored with vaginal smears for three consecutive cycles. The phase of the estrous cycle was determined by examining the cell types and amount of cells present according to an established method (Goldman et al, 2007, Develop Reprod Toxicol.). To eliminate effects caused by hormone level fluctuation, the intact rats used in the experiments were subjected to tMCAO on either the day of estrus or diestrus, when levels of circulating estrogen and progesterone are low compared to the proestrus phase.

Transient Unilateral Middle Cerebral Artery Occlusion

At 12 weeks of age and after 3 weeks of hormone treatment, female Wistar rats were subjected to transient unilateral middle cerebral artery occlusion (tMCAO) using an intraluminal occlusion technique (Stenman et al, 2002, Stroke). Anesthesia was induced by 4.5% isoflurane in N₂O:O₂ (70:30) and maintained by inhalation of 1.5-2.0% isoflurane in N₂O:O₂ (70:30) during the procedure. Mean arterial blood pressure, pCO₂, pO₂, pH and plasma glucose were measured prior to the occlusion through an arterial tail catheter (Radiometer; LabChart). A rectal thermometer connected to a homoeothermic blanket was used to maintain body temperature at +37° C. during the surgical procedure. An incision was made in the midline of the neck exposing the right common, internal and external carotid arteries. The common and external carotid arteries were permanently ligated by sutures and an incision was made in the common carotid artery. A laser Doppler probe (Perimed, Järfälla, Sweden) was fixed to the thinned skull in the area corresponding to the area supplied by the middle cerebral artery (1 mm posterior to bregma and 6 mm to the right from the midline). A silicone, rubber-coated monofilament (Doccol Corporation, Redlands, Calif., USA) was inserted through the incision until the tip reached the entrance of the right middle cerebral artery (MCA). The occlusion was confirmed by an abrupt reduction of cortical blood flow that was observed using laser Doppler monitoring. After securing the filament, the skin was sutured and anesthesia was discontinued. After two hours of occlusion, the rats were briefly re-anesthetized to remove the filament and allow reperfusion. Proper reperfusion was confirmed by a significant increase in blood flow as indicated by laser Doppler flowmetry. The animals were allowed to recover 48 hours after surgery with free access to food and water before they were anesthetized with CO₂ and decapitated. The brains were removed and immediately chilled in ice-cold bicarbonate buffer solution (for composition, see Drugs, Chemicals and Solutions). Right (occluded) and left (non-occluded) middle cerebral arteries were dissected free from adhering tissue and used for myograph studies.

Organ Culture

12-week old female rats were ovariectomized and implanted with a silastic capsule containing 17β-estradiol (n=6) as described above. Ovariectomized placebo-treated rats (n=6) and intact rats (n=6) were used for comparison. Three weeks after ovariectomy and hormone capsule implantation; the animals were anesthetized with CO₂ and decapitated. The brains were immediately removed and chilled in ice-cold bicarbonate buffer solution (for composition, see Drugs, Chemicals and Solutions). The MCAs were removed and studied with myography immediately or following 24 hours in organ culture with Dulbecco's modified Eagle's medium (DMEM; Gibco, Invitrogen, Carlsbad, Calif., USA) supplemented with penicillin (100 U ml⁻¹), streptomycin (100 μg mL⁻¹) and amphotericin B (0.25 μg mL⁻¹) at +37° C. in humidified 5% CO₂ in air.

In Vitro Pharmacology

Contractile properties of middle cerebral arteries were examined using a wire Mulvany-Halpern myograph that records isometric tension (Danish Myo Technology A/S, Aarhus, Denmark). Arteries were cut into cylindrical segments (2 mm), and mounted in the myographs by two parallel 40 μm wires inserted through the lumen. The myograph baths contained 5 ml +37° C. bicarbonate buffer solution (for composition, see Drugs, Chemicals and Solutions) continuously aerated with 5% carbon dioxide in oxygen resulting in pH 7.4. After a 20-minute equilibration period, the arteries were stretched to 90% of their normal internal circumference with a micrometer screw connected to one of the wires, corresponding with the size of the artery during physiological conditions with a transmural pressure of 100 mm Hg. The other wire was connected to a force displacement transducer attached to an analogue-digital converter (AD Instruments, Chalgrove, UK). The results were recorded on a computer using a Power Lab unit (AD instruments) and the software LabChart (ADInstruments). After the normalization procedure, the arteries were allowed to equilibrate at this tension for 20 min.

The contractile capacity was tested by switching the buffer to 5 ml potassium-rich buffer (63.5 mM, see Drugs, Chemicals and Solutions). The maximum potassium-mediated contraction was used as a reference value (=100%) for contractile capacity. To eliminate endothelial influence, the production of nitric oxide and prostaglandins was blocked with 100 μM L-NG-nitroarginine methyl ester (L-NAME) and 10 μM indomethacin, respectively, which were present in the tissue baths throughout the experiments on the arteries from the in vivo stroke model.

Receptors for 5-hydroxytryptamine receptors (5-HT) were evaluated by adding 5-carboxamidotryptamine (5-CT, a non-selective 5-HT₁ agonist) in concentrations ranging from 10⁻¹¹ to 10⁻⁵M (Hansen-Schwartz). The angiotensin type 1 (AT₁) receptor was evaluated by cumulative applications of Angiotensin II in concentrations ranging from 10⁻¹² to 10⁻⁶ M. 30 minutes prior to the experiment, the AT₂ receptor antagonist PD123319 was added to eliminate any AT₂ receptor-mediated effects. The selective ET_{B} receptor agonist sarafotoxin 6c (S6c) (Alexis Biochemicals, Farmingdale, N.Y., USA) was added in concentrations ranging from 10⁻¹¹ to 10⁻⁷ M.

Analysis and Statistics

The non-parametric Mann Whitney's test was used when comparing the maximum contraction (Emax) of two groups, and the Kruskal-Wallis test followed by Dunn's multiple comparison test was used when comparing three or more groups. Statistical analyses with p-values below 0.05 were considered significant. Results are expressed as mean±SD, and n=number of animals in the groups.

Drugs and Solutions

All substances were purchased from Sigma-Aldrich (St Louis, Mo., USA) if not stated otherwise. The bicarbonate buffer had the following composition: 119 mM NaCl, 15 mM NaHCO₃, 4.6 mM KCl, 1.5 mM CaCl₂, 1.2 mM NaH₂PO₄, 1.2 mM MgCl and 5.6 mM glucose. The bicarbonate buffer solution containing 63.5 mM K⁺ was obtained by partial exchange of NaCl for KCl in the above buffer.

Results

In Vitro Pharmacology

The maximum contractile responses induced by high potassium (63.5 mM) did not differ significantly among the treatment groups, between arteries from the occluded and non-occluded hemispheres, or between fresh and cultured arteries (the overall mean contraction was 4.2 mN). In each artery segment, the maximum potassium-induced response was used as a reference for contractile capacity (=100%) to compare the alterations in responses.

Vasoconstrictor Responses After tMCAO: Effects of Ovariectomy

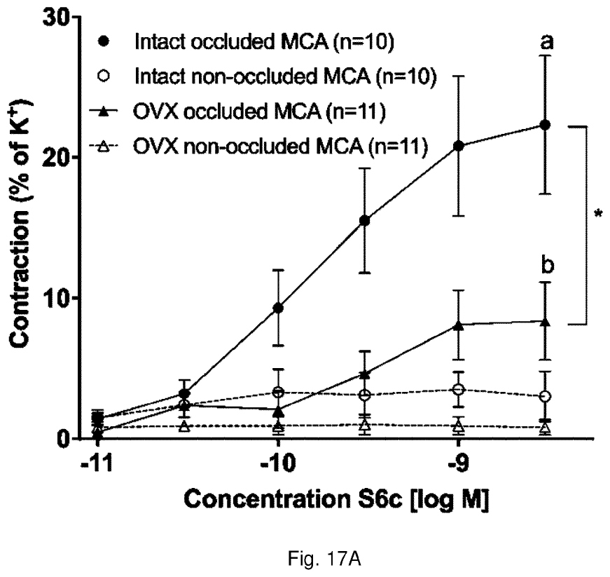

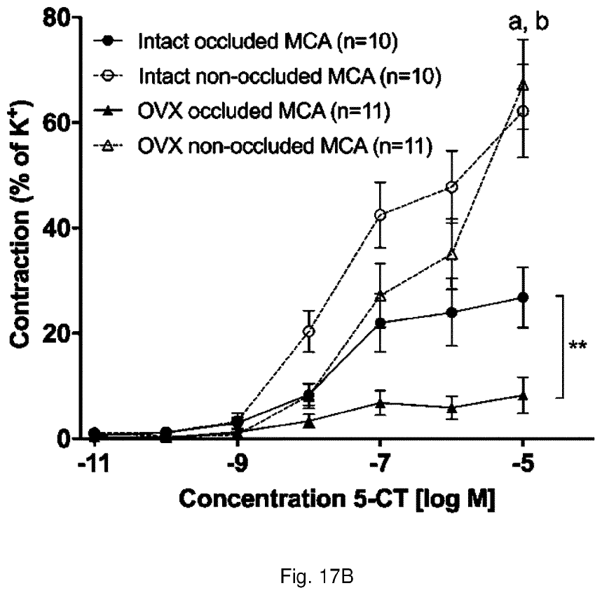

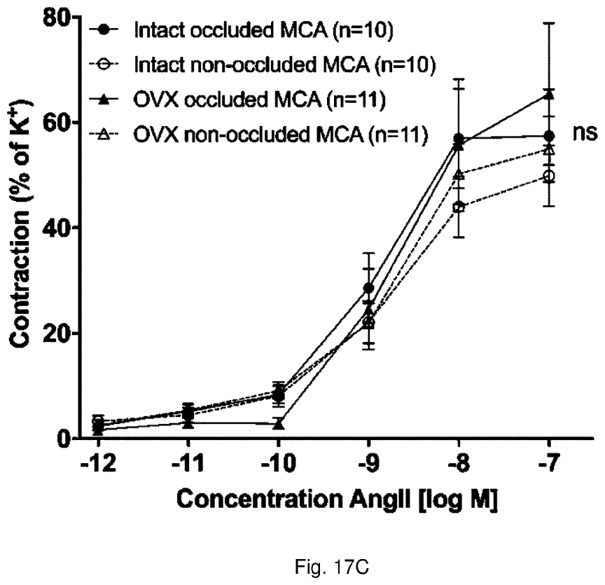

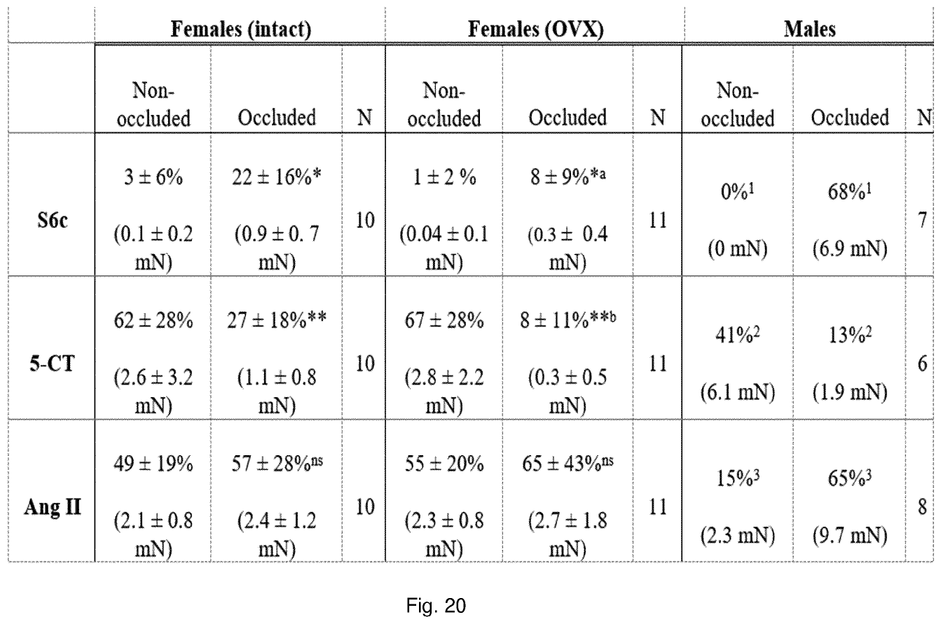

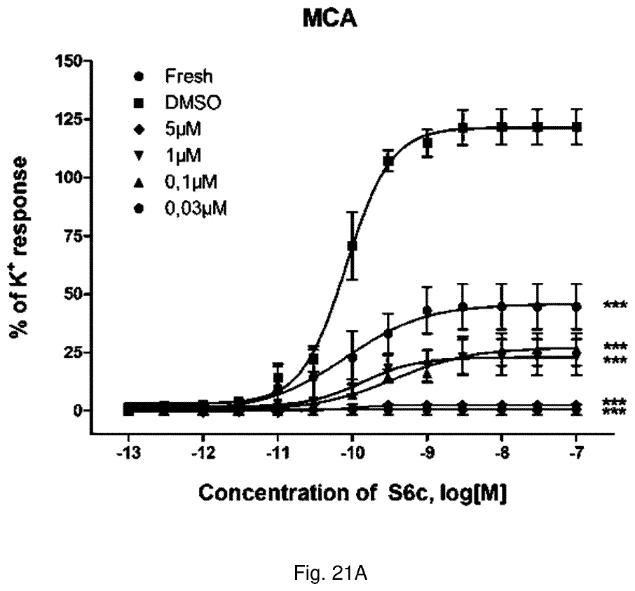

Female MCAs were examined 48 hours after the cerebral ischemia in a wire myograph. In arteries from the non-occluded side of the brain, there was little to no contraction in response to the selective ET_{B} receptor agonist S6c, as expected from previous studies. In contrast, the transiently occluded arteries showed significant contraction in response to S6c (FIG. 17A, FIG. 20). S6c-mediated contraction was significantly lower in the MCAs from ovariectomized females compared to intact females (8±9% and 22±16%, respectively; p<0.05) (FIG. 17A, FIG. 20).

Concentration-response curves for the 5-hydroxytryptamine 5-HT) receptor agonist 5-CT were similar in non-occluded arteries from intact and ovariectomized females (FIG. 17B, FIG. 20). The concentration-response curves in non-occluded arteries were biphasic, indicating 5-CT acted on more than one type of contractile 5-HT receptor in the artery, as shown previously in male cerebral arteries. In occluded arteries from intact females, 5-CT-mediated vasocontraction was in general significantly lower as compared to non-occluded arteries, and the curve was monophasic, consistent with a single receptor subtype. Interestingly, the arteries from ovariectomized animals showed almost no vasoconstrictor response towards 5-CT (8±11%), which differs significantly (p<0.01) from the response in intact females (27±18%) (FIG. 17B, FIG. 20). Hence, the mean reduction of 5-CT mediated response after tMCAO was greater in the ovariectomized rats compared to the intact females.

AT₁ receptor-mediated contraction to Angiotensin II (Ang II) was similar in occluded and non-occluded arteries (FIG. 17C, FIG. 20). This finding differs considerably from historical data in males where the AT₁-mediated contractility in the non-occluded artery was found to be relatively low compared to the occluded artery (FIG. 20). Ovariectomy did not affect the strong AT₁-receptor mediated contraction observed in occluded and non-occluded arteries (FIG. 17C, FIG. 20).

Vasocontractile Responses After tMCAO: Effects of 17β-Estradiol and Progesterone Treatment

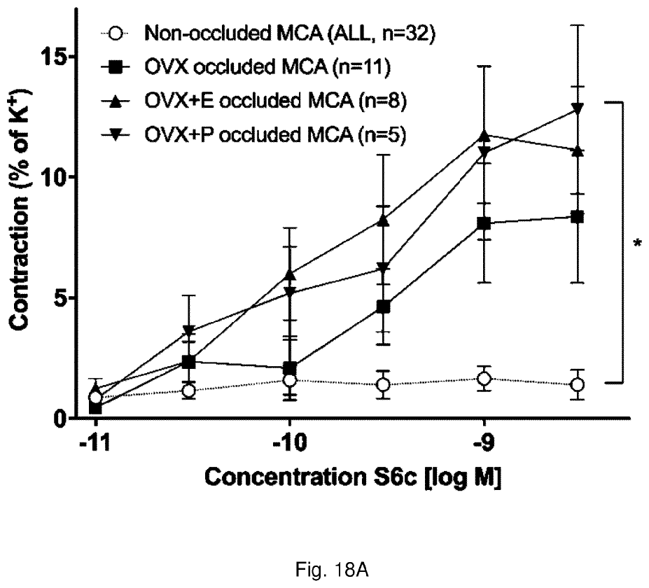

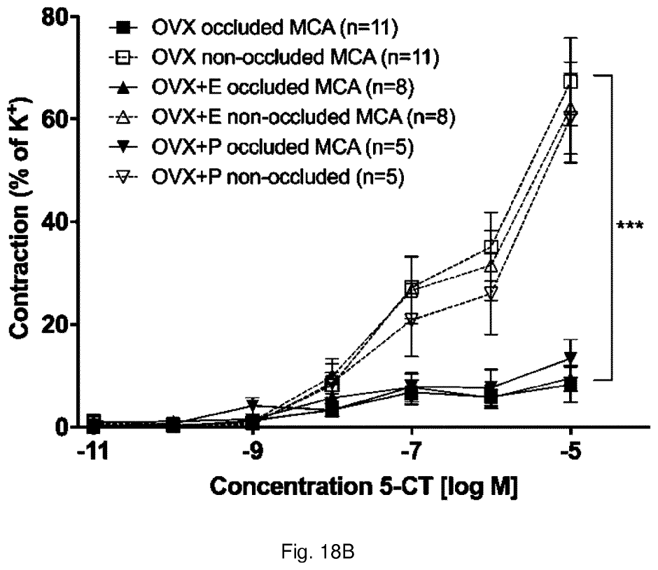

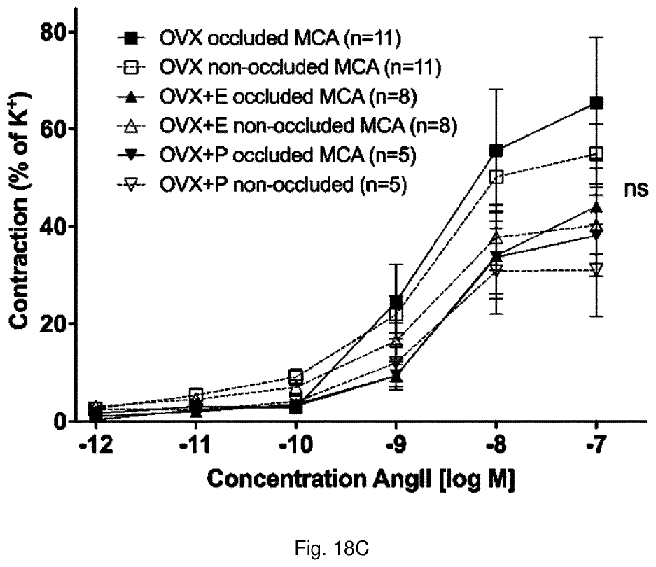

Ovariectomized rats were treated for 3 weeks with 17β-estradiol, progesterone or placebo via implanted capsules and then subjected to unilateral tMCAO. In general, the maximum contractile responses of occluded and non-occluded arteries to S6c, Ang II or 5-CT were not affected by the hormone treatments in comparison to arteries from placebo-treated ovariectomized rats (FIG. 18A-C). As shown in FIG. 17, ovariectomy resulted in significantly lower ET_{B}- and 5-HT-receptor mediated maximum contractile responses as compared to that seen in intact females while there was no differences in the already strong AT₁ receptor mediated response. Thus, maintaining a physiological level of progesterone or estrogen after ovariectomy was not enough to prevent the reduction of maximum contractile response towards ET_{B} and 5-HT receptor agonists.

Vasomotor Responses Following Organ Culture in Arteries from Intact, Ovariectomized and 17β-Estradiol-Treated Females

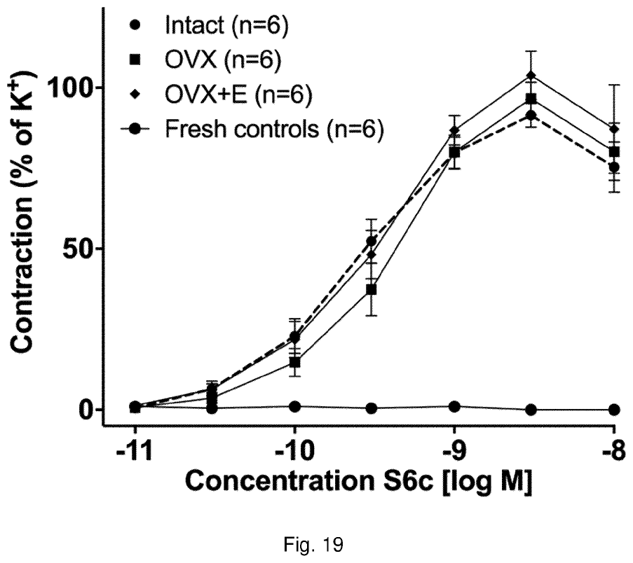

MCA segments from OVX, OVX+E and intact female rats were studied in the wire myograph immediately after isolation or following 24 h of organ culture. ET_{B} receptor mediated contraction was elicited by cumulative application of S6c. There was no S6c mediated contraction in fresh arteries (fresh control); however, a strong contraction to increasing concentrations of S6c was seen in all cultured arteries, and this response did not differ among the treatment groups (FIG. 19). Thus, prior exposure to ovarian hormones in vivo did not affect the upregulation of ET_{B} receptors that occurred in the arteries during culture.

Conclusion

The maximum contractile response mediated by the endothelin B (ET_{B}) receptor agonist sarafotoxin 6c (S6c) was increased in female arteries after I/R, but the maximum response was significantly lower in MCAs from ovariectomized females.

In contrast, the maximum contraction mediated by the 5-hydroxytryptanime receptor agonist 5-carboxamidotryptamine (5-CT) was reduced after I/R, with arteries from ovariectomized females showing a greater decrease in maximum contractile response. Contraction elicited by angiotensin II was not altered by any procedure. Supplementation with either estrogen or progesterone in ovariectomized females did not modify I/R-induced changes in ET_{B} and 5-CT induced vasocontraction. Isolated MCAs subjected to organ culture exhibited an increase in ET_{B}-mediated contraction. Responses were similar in arteries cultured from intact, placebo-treated ovariectomized and estrogen-treated ovariectomized females. These findings suggest that sex hormones do not directly influence vasocontractile receptor alterations that occur after ischemic stroke; however, ovariectomy does impact this process.

ET_{B} receptor upregulation was more pronounced in males than in females after tMCAO. In contrast, the contractile responses to 5-CT and Ang II in non-occluded female MCAs were stronger than in males: After tMCAO the 5-CT responses were reduced in both gender and the Ang II responses unaltered in females and increased in males. Thus, the vascular responses behaves somewhat different depending on sex (FIG. 20).

The hypothesis was that these male-female differences reflect an effect of sex hormones on contractile responses in cerebral arteries. Surprisingly, while ET_{B}-mediated maximum contraction was increased following tMCAO and after organ culture, no effect was found of sex hormone replacement after ovariectomy. On the contrary, in female arteries following tMCAO, vasocontractile responses to S6c and 5-CT are markedly lower than previously reported for males. Most interestingly there was a significant effect of ovariectomy on vasocontractile receptor responses after tMCAO that was not reversed by either estrogen or progesterone replacement. If anything this would imply a gender difference in handling the effect of a stroke, being more favorable in females than in males.

### Example 11

### Systemic Administration of the MEK1/2 Inhibitor Trametinib (GSK1120212) After In Vivo Subarachnoid Hemorrhage in Rats—Comparison with U0126

Materials and Methods

Animals

Male Sprague-Dawley rats (300-350 g, Taconic, Denmark) were used. All procedures were performed strictly within national laws and guidelines and were approved by the Danish Animal Experimentation Inspectorate (2012-15-2934-389).

In Vivo Subarachnoid Hemorrhage

SAH was induced as described in detail before (Povlsen et al, 2013, BMC Neurosci) with the exceptions that rats were anesthetized with a 2.5 mL·kg-1 mixture of hypnorm-midazolam (1:1:2) in sterile water and 300 μl of blood was injected to the chiasma opticum.

Experimental Groups

Thirty-two rats were operated for this study. Animals were treated intraperitoneally with 250 μl/body weight of a 1 mM solution of trametinib (Selleckchem), yielding a final dose of 95 μg/body weight, diluted in 10% cremophor and 10% PEG400 in NaCl. Animals in the vehicle and sham group were treated with 250 μl/body weight 10% cremophor and 10% PEG400 in NaCl. Treatment was intraperitoneally administered either at 1 h and 24 h or 6 h and 24 h post surgery. Animals were terminated 48 h after surgery by CO2 anesthesia and decapitation.

In Vitro Organ Culture

MCAs from naïve rats were dissected and segments (1.5 mm) were incubated for 48 h in Dulbecco's modified Eagle's medium contained L-glutamine (584 mg/L) supplemented with penicillin (100 U/ml) and streptomycin (100 mg/ml) at humidified 5% CO2 atmosphere. Before incubation, 4 different concentrations of trametinib (5 μM, 1 μM, 0.1 μM or 0.03 μM), dissolved in 0.1% dimethyl sulfoxide (DMSO) in NaCl, or 0.1% DMSO in NaCl (vehicle) was added.

Wire Myography

A wire myograph was used to record the isometric tension in segments (1.5 mm) of isolated cerebral arteries. Vessel segments received an initial pretension of 2 mN/mm and were precontracted with a solution of 63.5 mM K+. Only basilar arteries (BAs) with K+-induced responses over 2 mN and middle cerebral arteries (MCAs) with K+-induced responses over 0.7 mN were used for experiments. Concentration-response curves were obtained by cumulative application of Sarafatoxin 6c (S6c), an ETB receptor-specific agonist in the concentration range 10⁻¹² to 10⁻⁴ M (Alexis Biochemicals, USA) and Endothelin-1 (ET-1) in the concentration range 10⁻¹⁴ to 10⁻⁷ M (AnaSpec, USA).

Intracellular Flow Cytometry

The MCAs, BA and the circle of Willis were pooled from one rat. The VSMCs of the cerebral arteries was isolated by a novel technique advanced by the present inventors based on two other protocols (Navone et al, 2013, Nat Protoc; van Beijnum et al, 2008, Nat Protoc). The tissue were disrupted mechanically (scalpel) and then subjected to enzymatic digestion with highly purified Collagenase I and Collagenase II. Isolated cell suspensions were fixed with 4% paraformaldehyde for 30 minutes, washed with PBS and thereafter permeabilized with 0.25% TritonX-100. Cells were resuspended in blocking buffer containing 5% donkey serum and double-stained overnight at 4° C. with primary goat anti-SM22α (1:100, Abcam) or goat isotope control IgG (5 μg/mL, Abcam) and primary antibody rabbit anti-ET_{B} (1:100, Abcam) or rabbit isotope control IgG (10 μg/mL, Abcam) in the same blocking buffer. Next day, cell samples were incubated with Alexa 488-conjugated donkey anti-goat IgG (1:100, Jackson ImmunoResearch) and Allophycocyanin (APC)-conjugated donkey anti-rabbit IgG (1:100) for 2 h (dark) at RT. Finally, cell suspensions were diluted to a final volume of 0.5 mL with PBS before analyzed by fluorescent-activated cell sorting (FACS) on the BD FACSVerse machine (BD Biosciences, USA). Fluorescence was induced with a 640 nm red laser. The ratio of SM22α-positive cells expressing ET_{B} was calculated in each sample. Data was analyzed by the BD FACSuite Software.

Calculations and Statistics

Data are presented as means±SEM, n refers to the number of rats. Concentration-contraction curves were compared to two-way ANOVA. For normalization, K⁺-evoked contractile responses was set to 100%. Flow cytometry were analyzed with one-way ANOVA. Significance level was set to p<0.05.

Results

In all rats, physiological parameters and temperature were within acceptable limits during surgery without any differences between groups. ICP increased from 5.4 mmHg to 116.8 mmHg and cortical CBF dropped to 19% of resting flow (average values for all SAH animals).

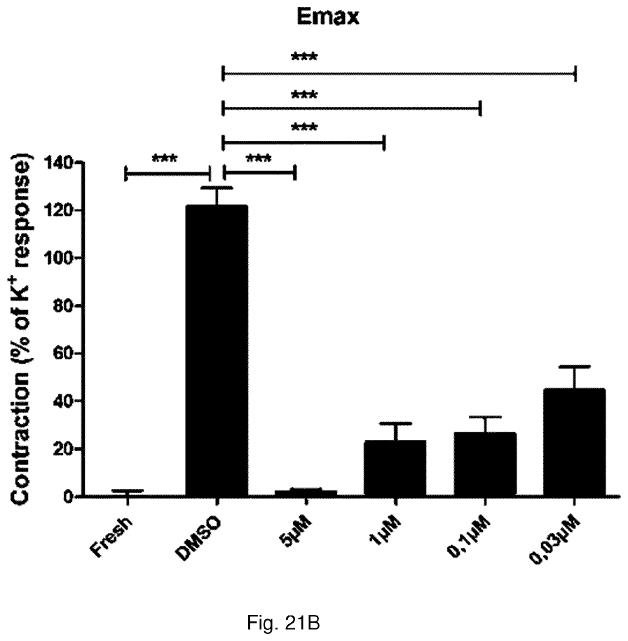

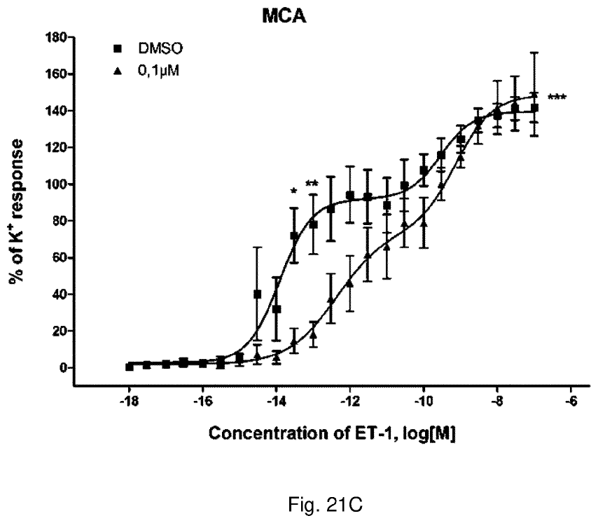

In the initial, in vitro experiments, freshly isolated MCAs (controls) showed no contractile response to the ET_{B} receptor agonist S6c. After 48 h of OC, S6c yielded a strong contractile response in MCAs incubated with vehicle. However, co-incubation with trametinib significantly inhibited the S6c-induced contraction 48 h after OC in a concentration-dependent manner (FIG. 21A. The maximal contraction (Eₘₐₓ) induced by S6c, in all groups, is shown in FIG. 21B. Based on the above mentioned results, 0.1 μM of trametinib confirmed the inhibitory effect on increased ET-1-induced vasoconstriction (FIG. 21C). In vehicle incubated MCAs, the enhancement was observed as leftward shifts of the ET-1concentration-response curve with a transition into biphasic curve shape. MCAs incubated in the presence of trametinib resulted in a right-ward shift of the concentration-response curve, indicating a total blockage of the ET_{B} receptor subtype response.

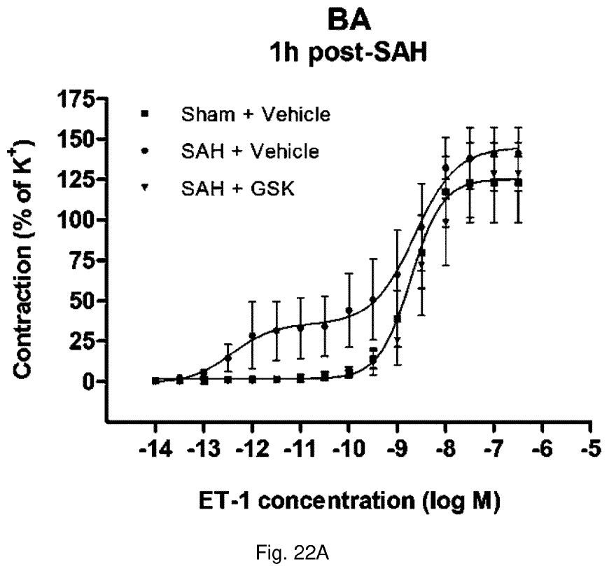

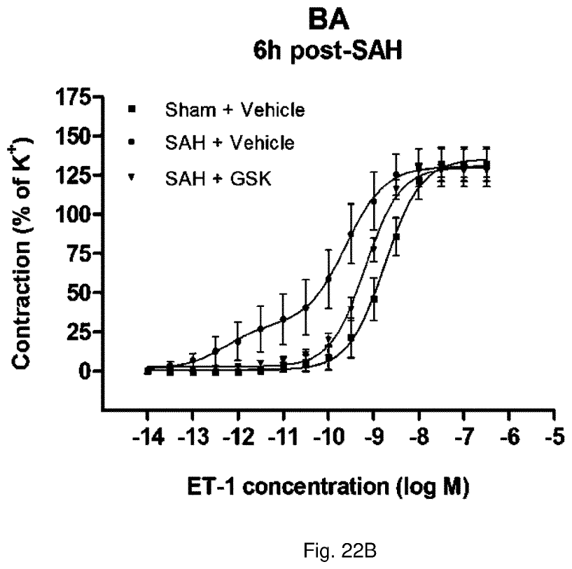

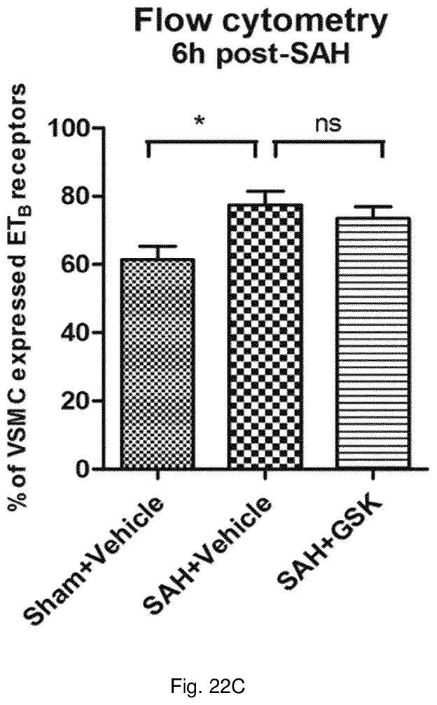

To confirm in vivo the effect of the trametinib treatment on SAH-induced increased ET-1 mediated vasoconstriction, two different treatment approaches were used; intraperitoneal administration of 1 mM trametinib at 1 and 24 h (FIG. 22A) or at 6 and 24 h (FIG. 22B) post-SAH. The increased ET-1-induced concentration-response curve in BAs after SAH was observed as leftward shifts and were significantly inhibited by trametinib using both treatment approaches. The enhanced contractile responses observed after the 6 h post-SAH treatment was verified with protein analyses using flow cytometry. There was a significant increase of SMC expressing ET_{B} receptor after SAH (vehicle) (74.2%±12.2%; n=7) compared to sham (61.4%±10.2%; n=6). However, this increased protein expression was not significant abolished by trametinib with this particular treatment approach (FIG. 22C).

Conclusion

The MEK1/2 inhibitor trametinib is a potent compound with the ability to completely inhibit the increased ET_{B}-receptor mediated contraction in cerebral arteries after in vitro OC. trametinib can be administered systemically in vivo to the rats but still diminish the increased ET-1 mediated vasoconstriction, in cerebral arteries 48 h after SAH, in the same proportional as after intracisternally administration.

The initial in vitro experiments showed that 0.1 μM trametinib was able to significantly inhibit the increased ET_{B}-receptor mediated contraction induced by ET-1. In former studies using the same in vitro setup, a concentration of 10 μM U0126 was used for equivalent inhibition. U0126 has previously been in vivo administered intraperitoneally in models of global cerebral ischemia and focal cerebral ischemia with a final concentration of 50 mM dissolved in 100% DMSO. However, intraperitoneally administration has never been used in earlier studies of SAH, but rather intracisternally administration of U0126 (10 μM dissolved in 0.1% DMSO) at 6, 12, 24 and 36 hours post-SAH. In the current study the potent and selective MEK1/2 inhibitor trametinib was dissolved in a non-DMSO solution (cremophor/PEG400 in NaCl) and rats were treated intraperitoneally twice (1 and 24 or 6 and 24 hours post-SAH) with a final concentration of 1 mM. Under these conditions, the results demonstrate a positive effect of trametinib on increased vasoconstriction in the cerebral arteries 48 h after SAH.

In conclusions, the results demonstrate that the potent MEK1/2 inhibitor trametinib could be used by any treatment application for inhibition of increased vasoconstriction after SAH.

1. A MEK inhibitor of formula (I), 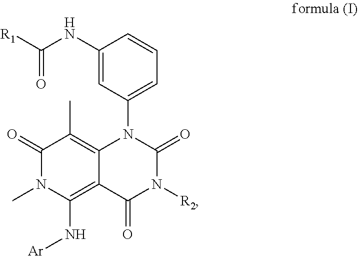
or a pharmaceutically acceptable salt thereof,
wherein;
R₁ is a C1-C6 alkyl, such as methyl,
R₂ is a C1-C6 alkyl, such as cyclopropyl,
Ar is selected from the group consisting of aryl and heteroaryl;
for use in the prevention or treatment of a stroke in a subject.

2. The MEK inhibitor according to claim 1, wherein R₁ is a C1-C3 alkyl.

3. The MEK inhibitor according to any one of the preceding claims, wherein R₁ is a linear C1-C3 alkyl.

4. The MEK inhibitor according to any one of the preceding claims, wherein R₁ is methyl or ethyl.

5. The MEK inhibitor according to any one of the preceding claims, wherein R₁ is methyl.

6. The MEK inhibitor according to claim 1, wherein R₂ is C2-C4 alkyl.

7. The MEK inhibitor according to any one of the preceding claims, wherein R₂ is C3 or C4 cycloalkyl.

8. The MEK inhibitor according to any one of the preceding claims, wherein R₂ is cyclopropyl.

9. The MEK inhibitor according to any one of the preceding claims, wherein Ar is phenyl or substituted phenyl.

10. The MEK inhibitor according to any one of the preceding claims, wherein Ar is substituted phenyl.

11. The MEK inhibitor according to any one of the preceding claims, wherein Ar is 2-fluoro-4-iodophenyl.

12. The MEK inhibitor according to any one of the preceding claims, wherein R₁ is a C1-C3 alkyl, R₂ is C2-C4 alkyl, and Ar is substituted phenyl.

13. The MEK inhibitor according to any one of the preceding claims, wherein R₁ is methyl or ethyl, R₂ is C3 or C4 cycloalkyl, and Ar is substituted phenyl.

14. The MEK inhibitor for use according to any one of the preceding claims, wherein the MEK inhibitor is of formula (II), 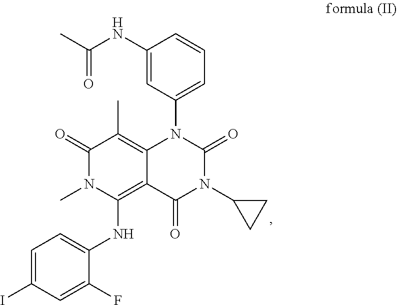 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt thereof.

15. The MEK inhibitor for use according to any one of the preceding claims, wherein the stroke is selected from the group consisting of: ischemic stroke, haemorrhagic stroke, and transient ischemic attack.

16. The MEK inhibitor for use according to any one of the preceding claims, wherein the stroke is selected from the group consisting of: global ischemia and focal ischemia.

17. The MEK inhibitor for use according to any one of the preceding claims, wherein the ischemic stroke results from an embolism, thrombosis, systemic hypoperfusion, cerebral venous sinus thrombosis, a sudden drop in blood pressure or heart stop, rupture of a cerebral artery or arteriole, or a combination thereof.

18. The MEK inhibitor for use according to any one of the preceding claims, wherein the haemorrhagic stroke results from intracerebral haemorrhage, subarachnoid haemorrhage, or a combination thereof.

19. The MEK inhibitor for use according to any one of the preceding claims, wherein the intracerebral haemorrhage is intraparenchymal, intraventricular, or a combination thereof.

20. The MEK inhibitor for use according to any one of the preceding claims, wherein the stroke results from subarachnoid haemorrhage (SAH).

21. The MEK inhibitor for use according to any one of the preceding claims, wherein the stroke results from Ischemic Brain Injury (TBI).

22. The MEK inhibitor for use according to any one of the preceding claims, wherein the stroke is a delayed cerebral ischemia (DCI).

23. The MEK inhibitor for use according to any one of the preceding claims, wherein the DCI presents with inflammation, oedema, delayed cerebral vasospasm (CVS), blood-brain barrier disruption and/or increase in contractile receptor expression, such as those for endothelin, angiotensin, serotonin and thromboxane or prostaglandins.

24. The MEK inhibitor for use according to any one of the preceding claims, wherein the MEK inhibitor is administered to the subject without surgery prior to, concurrent with, or subsequent to the administration.

25. The MEK inhibitor for use according to any one of the preceding claims, wherein the MEK inhibitor is administered to the subject prior to, concurrent with, or subsequent to thrombectomy.

26. The MEK inhibitor for use according to any one of the preceding claims, wherein the MEK inhibitor is administered to the subject prior to, concurrent with, or subsequent to thrombolysis.

27. The MEK inhibitor for use according to any one of the preceding claims, wherein the MEK inhibitor is administered to the subject prior to, concurrent with, or subsequent to a surgical procedure selected from the group consisting of: coiling and clipping.

28. The MEK inhibitor for use according to any one of the preceding claims, wherein the MEK inhibitor is administered to the subject prior to, concurrent with, or subsequent to a neuroradiological procedure.

29. The MEK inhibitor for use according to any one of the preceding claims, wherein the MEK inhibitor reduces or prevents reperfusion damage resulting from the neuroradiological procedure.

30. The MEK inhibitor for use according to any one of the preceding claims, wherein the MEK inhibitor is administered to the subject before it has been determined if the subject suffers from an acute ischemic stroke or a haemorrhagic stroke.

31. The MEK inhibitor for use according to any one of the preceding claims, wherein the MEK inhibitor is administered orally, intrathecally, intraperitoneally, intraocularly, intranasally, or intravenously.

32. The MEK inhibitor for use according to any one of the preceding claims, wherein the MEK inhibitor is administered intravenously.

33. The MEK inhibitor for use according to any one of the preceding claims, wherein the MEK inhibitor is administered to the subject up to 6 hours subsequent to the onset of the stroke, such as up to 1 hour, such as up to 2 hours, such as up to 3 hours, such as up to 4 hours, such as up to 5 hours subsequent to the onset of the stroke.

34. The MEK inhibitor for use according to any one of the preceding claims, wherein the MEK inhibitor is administered one or more times daily for up to 3 days subsequent to the onset of the stroke.

35. The MEK inhibitor for use according to any one of the preceding claims, wherein the subject is a human subject.

36. Use of a MEK inhibitor of formula (I) as defined in any one of the preceding claims, for:
a. reducing endothelin-1 induced contractility;
b. increasing endothelin B receptor function; and/or
c. improving neurological score, which may be evaluated by a subject's ability to traverse a rotating pole, after induced subarachnoid haemorrhage.

37. A method of treating or reducing the risk of developing a stroke in a subject, wherein the method comprises the steps of administering a MEK inhibitor of formula (I), 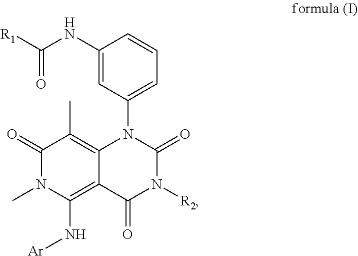
or a pharmaceutically acceptable salt thereof,
wherein;
R₁ is a C1-C6 alkyl, such as methyl,
R₂ is a C1-C6 alkyl, such as cyclopropyl,
Ar is selected from the group consisting of aryl, phenyl, and heteroaryl;
to a subject in need thereof, thereby treating or reducing the risk of developing a stroke.

38. A composition comprising, separately or together, the MEK inhibitor of formula (II), 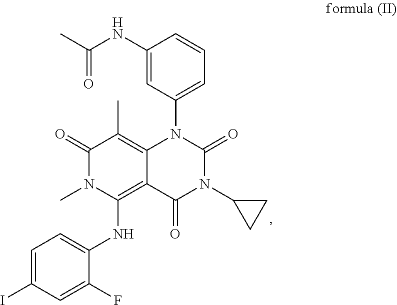 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt thereof, and a further medicament.

39. The composition according to any one of the preceding claims, wherein the further medicament is selected from the group consisting of: a calcium channel blocker, such as Nimodipine, and an endothelin receptor (ET) receptor blocker, such as clazosentan.

40. A kit of parts comprising;
a MEK inhibitor as defined in any one of the preceding claims; and
a further medicament as defined in any one of the preceding claims;
wherein the MEK inhibitor and the further medicament are formulated for simultaneous or sequential use; and optionally instructions for use.
